(19) United States
(12) Patent Application Publication
(10) Pub. No.: US 2024/0263158 A1
(43) Pub. Date: Aug. 8, 2024

(54) DUPLEX-SPECIFIC DNASES

(71) Applicant: New England Biolabs, Inc., Ipswich, MA (US)

(72) Inventors: Lisa L. Maduzia, Topsfield, MA (US); Ashley Luck, Ipswich, MA (US); Heidi Crosby, Ipswich, MA (US); Mehul Ganatra, Gloucester, MA (US); Pei-Chung Hsieh, Topsfield, MA (US); Vladimir Potapov, Auburndale, MA (US); Sean R. Johnson, Beverly, MA (US); Eric J. Cantor, Topsfield, MA (US); Jennifer Ong, Salem, MA (US); Thomas C. Evans, Jr., Topsfield, MA (US)

(73) Assignee: New England Biolabs, Inc., Ipswich, MA (US)

(21) Appl. No.: 18/390,303

(22) Filed: Dec. 20, 2023

Related U.S. Application Data
(60) Provisional application No. 63/482,696, filed on Feb. 1, 2023.

(51) Int. Cl. C12N 9/22; C12Q 1/6818

(52) U.S. Cl. CPC C12N 9/22; C12Q 1/6818

(57) ABSTRACT

The present disclosure relates, according to some embodiments, to enzymes, systems, compositions, methods, apparatus, and workflows for selectively cutting, cleaving, digesting and/or hydrolyzing duplex DNA strands or the DNA strand of a DNA/RNA hybrid.

### CROSS-REFERENCE TO RELATED APPLICATIONS

This application claims priority to U.S. Provisional Application No. 63/482,696 filed Feb. 1, 2023. The contents of which are hereby incorporated in its entirety by reference.

### SEQUENCE LISTING STATEMENT

This disclosure includes a Sequence Listing submitted electronically in XML format under the file name “NEB-458.xml” created on Dec. 18, 2023, having a size of 62.0 KB. This Sequence Listing is incorporated herein in its entirety by this reference.

### BACKGROUND

The chemical stability of DNA supports its role as a molecular repository of genetic information. This stability can be undesirable in certain contexts where, for example, its persistence can contribute to the genesis and/or pathology of cancer and other diseases. Its presence can also impair sample analysis, for example, when studying RNA. DNA's stability is limited—it is subject to damage and many chemical and biological agents exist which can digest it. For example, phosphodiester linkages in the backbone of single-stranded or double-stranded DNA may be hydrolytically cleaved by DNases. Type I DNases have a pH optimum near neutral, require divalent cations, and produce 5′-phosphate deoxynucleotide products from DNA cleavage. Type II DNases have an acidic pH optimum, may be activated by divalent cations, and produce 3′-phosphate deoxynucleotide products from DNA cleavage.

### SUMMARY

The present disclosure provides systems, compositions, methods, apparatus, and workflows that include dsDNases with desirable properties including, for example, selective digestion of duplex DNA and/or selective digestion of the DNA strand of a DNA/RNA hybrid, sufficient solubility (and other biochemical properties) to support production in bacteria and/or yeast, and capacity to bind magnesium (e.g., in or near the active site). Example embodiments of dsDNases with desirable properties include engineered dsDNases having an amino acid sequence that is at least 80% identical, at least 85% identical, at least 88% identical, at least 90% identical, at least 92% identical, at least 95% identical, at least 98% identical, and/or at least 99% identical to one or more of SEQ ID NOS: 1-13, 19, 28 and 29. In some embodiments, a dsDNase may have an amino acid sequence that is at least 95% identical, at least 98% identical, at least 99% identical, and/or 100% identical to SEQ ID NO: 14-18.

The present disclosure relates, in some embodiments, to an engineered dsDNase having an amino acid sequence that is ≥85% identical (optionally, ≥90% identical) to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO: 12, or SEQ ID NO:13. An engineered dsDNase may have an amino acid sequence that is, for example, at least 95% identical to SEQ ID NO: 1 or SEQ ID NO:2. An engineered dsDNase may have an amino acid sequence at least 97% identical to the amino acid sequence of SEQ ID NO: 19, SEQ ID NO:28, or SEQ ID NO:29. An engineered dsDNase may have an amino acid sequence identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 19, SEQ ID NO:28, or SEQ ID NO:29.

A dsDNase in the presence of 50 mM NaCl, according to some embodiments, may have at least 50% of its peak activity in the absence of NaCl. In some embodiments, a dsDNase in the presence of 100 mM NaCl may have at least 20% of its peak activity in the absence of NaCl. The present disclosures relate to compositions, for example, compositions comprising an engineered dsDNase and one or more salts, for example, wherein the composition has a total salt concentration of at least 50 mM. In some embodiments, a composition may comprise a dsDNase and one or more DNA hydrolysis products.

A composition, in some embodiments, may comprise one or more proteins and/or enzymes other than the dsDNase. The present disclosure provides, in some embodiments, fusion proteins. For example, a fusion protein having a single polypeptide chain where the single polypeptide chain comprises a dsDNase and at least one of an affinity tag, a secretion signal, and a linker.

The present disclosure further relates to methods of contacting a composition with a dsDNase (e.g., a dsDNase having an amino acid sequence that is ≥85% identical to any of SEQ ID NO: 1-13, and 19 or that is ≥85% identical to any of SEQ ID NO: 14-18). A composition to be contacted with a dsDNase may be or may comprise, for example, a nucleic acid library, one or more products of a nucleic acid synthesis reaction (e.g., a transcription reaction or an amplification reaction), an environmental sample, a biological fluid, a tissue, a pharmaceutical preparation, a biological specimen or extract, a test sample, or any other material having or potentially having a dsDNase substrate and/or a target sequence capable of forming a dsDNase substrate when contacted with a target probe having a sequence complementary to the target sequence. In some embodiments, a method for depleting a target sequence from a population of duplex DNA molecules, the population comprising at least one copy of a DNA strand having the target sequence may comprise (a) hybridizing to the at least one strand of a polynucleotide having a sequence complementary to the target sequence to form a target duplex, and (b) contacting the target duplex with a dsDNase according to claim 1 to form a target sequence depleted population of duplex DNA molecules and DNA hydrolysis products. According to some embodiments, the polynucleotide having a sequence complementary to the target sequence is an oligonucleotide probe, comprises RNA, and/or comprises DNA. The polynucleotide having a sequence complementary to the target sequence, in some embodiments, may be a hairpin probe comprising, in a 5′ to 3′ direction, a fluorophore, a first hairpin stem sequence, the sequence complementary to the target sequence, a second hairpin stem sequence complementary to the first hairpin stem sequence, and a quencher corresponding to the fluorophore. Contacting this material with a dsDNase may generate, in some embodiments. DNA hydrolysis products comprising the fluorophore and the quencher, wherein the fluorophore is sufficiently separated from the quencher to be fluorescently active. A target sequence may be a repetitive sequence present in the population of duplex DNA molecules (e.g., a genomic library). In some embodiments, hybridizing to at least one strand of a polynucleotide having a sequence complementary to the target sequence to form a target duplex may further comprise denaturing and reannealing the population of duplex DNA molecules and each target duplex comprises hybridized repetitive sequences.

In some embodiments, a method for hydrolyzing DNA (e.g., dsDNA) may comprise contacting (a) a composition comprising DNA and optionally RNA, and (b) a dsDNase (e.g., a dsDNase having an amino acid sequence that is ≥85% identical to any of SEQ ID NO: 1-13, 19, 28, and 29 or that is ≥85% identical to any of SEQ ID NO: 14-18) to form a reaction mixture comprising DNA hydrolysis products. A reaction mixture may have a total salt concentration of, for example, 0 mM to 150 mM. In some embodiments, the dsDNase may be specific for dsDNA. For example, where the composition comprises RNA, the reaction mixture retains at least 90% (optionally weight or molar percent) of the RNA in the starting composition (e.g., after a reaction time of at least 10 minutes, at least 20 minutes, at least 30 minutes). A reaction mixture may comprise less than 10% (optionally weight or molar percent) of the dsDNA present in the starting composition. According to some embodiments, a reaction mixture may comprise any desired buffer, for example, a Tris+magnesium buffer or a commercial buffer like NEB r1.1 (10 mM Bis-Tris-Propane-HCl, 10 mM MgCl₂, 100 μg/ml recombinant albumin, pH 7.0)(New England Biolabs, Inc., Ipswich, MA). In some embodiments, a composition to be contacted with a dsDNase may comprise one or more DNA:DNA duplexes (e.g., comprising no more than one mismatch per 10 paired nucleotides), one or more RNA:DNA duplexes, or both one or more DNA:DNA duplexes and one or more RNA:DNA duplexes. Optionally, duplex DNA in a composition may comprise repetitive DNA sequences.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1A shows an example of a dsDNase (SEQ ID NO:2) aligned to a dsDNase consensus sequence (SEQ ID NO:31) generated using ESPript (Robert, X. and Gouet, P. (2014)). Secondary structure assignments are based on a structure model of SEQ ID NO:2 generated using AlphaFold2 (Jumper et al., Nature, 2021; Mirdita et al., Nature Methods, 2022). Predicted beta strands (β), alpha helices (α), 3₁₀ helices (η), and turns (T) are indicated in the top line, and the second line shows residue numbering for Seq ID NO:2. Numbering under the cysteine residues corresponds to the disulfide bonds shown in FIG. 1B.

FIG. 1B is a representation of a three-dimensional structure of a dsDNase (SEQ ID NO:2) generated using AlphaFold2 and Pymol. Coils illustrate alpha helices, arrows illustrate beta pleated sheets (in the N to C direction) and lines representing turns and loops. Disulfide bonds connecting portions of the primary structure are shown in red and numbered according to the bottom line in the alignment of FIG. 1A.

FIG. 1C is a close-up view of the active site of a dsDNase (SEQ ID NO:2), including the catalytic magnesium ion shown in cyan. The catalytic ββα-metal motif is colored light green with stick representations of active site residues arginine 209, histidine 211, and asparagine 214 side chains. FIG. 1D shows a portion of the sequence of the same dsDNase (SEQ ID NO:2), indicating the beta strands and alpha helix that correspond to the ββα-metal motif shown in FIG. 1C. Beta strand and alpha helix numbering matches that shown in the alignment in FIG. 1A. Residues shown in bold are the active site residues arginine 209, histidine 211, and asparagine 214.

FIG. 2A shows an example of a fluorescent probe comprising a 35 nt DNA oligo with a 5′ FAM label and a 3′ quencher (Black Hole Quencher-1) (SEQ ID NO: 24). When the probe is heated to 95° C. and then gradually cooled, it folds into a hairpin structure in which the quencher and fluorophore are close enough to one another for the quencher to quench the fluorophore. Cleavage of the DNA hairpin allows FAM to separate from the quencher, resulting in an increase in fluorescence that can be measured at 517 nm using a spectrophotometer. Some screening reactions contained 3 μM probe in a 30 μl reaction, and reactions were carried out in black half-area 96-well plates (costar 3694).

FIG. 2B shows an example of a fluorescent probe comprising a single-stranded 15 nt DNA oligo (1 of many possible random sequences shown) with a 5′ FAM label and a 3′ quencher (Black Hole Quencher-1) (SEQ ID NO: 26). The short length of the oligo allows the quencher to quench the fluorophore. Cleavage of the ssDNA oligo releases FAM, resulting in an increase in fluorescence that can be measured at 517 nm using a spectrophotometer.

FIG. 2C shows an example of a fluorescent probe comprising a 60 nt linear DNA oligo with a 5′ FAM label (SEQ ID NO: 26).

FIG. 2D shows an example of a fluorescent probe comprising a single-stranded 50 nt DNA oligo (1 of many possible random sequences) with a 5′ ROX label (SEQ ID NO: 27).

FIGS. 3A, 3B, 3C, 3D, and 3E show examples of methods in which a dsDNase may be used.

FIG. 3A illustrates an example method comprising contacting a first (optionally longer) DNA strand and a second (optionally shorter) DNA strand to form a homoduplex comprising a duplex portion and one or more (e.g., two) overhangs. In some embodiments, the first strand or the second strand may comprise a DNA sequence complementary to a target sequence. For example, a second strand may be contacted with the first strand (or a library of first strands (each having the same or different sequences) to form a duplex with the target sequence (if present) in a first strand. A method may further comprise contacting the homoduplex with a dsDNase to form cleavage products. As illustrated, the dsDNase may cleave (digest) away both DNA strands of the duplex portion. In some embodiments, a first strand or a second strand may be linked to a surface (e.g., a bead, a plate, or a well), optionally through a linker.

FIG. 3B illustrates an example method comprising contacting a first (optionally longer) dsDNA comprising a top and bottom strand and a second (optionally shorter) dsDNA comprising a top and bottom strand to form a homoduplex comprising the top strand of the first DNA and bottom strand of the second DNA and a homoduplex comprising the bottom strand of the first DNA and the top strand of the second DNA. Each homoduplex may comprise a duplex portion and one or more (e.g., two) overhangs. A method may further comprise contacting one or both homoduplexes with a dsDNase to form cleavage products. Cleavage products may be treated (e.g., contacted with a dsDNase inhibitor, heated, and/or washed) to inactivate and/or remove the dsDNase and form treated cleavage products. Treated cleavage products may be annealed to form duplex DNA products.

FIG. 3C illustrates an example method comprising contacting a first (optionally longer) DNA strand (thick bar) and a second (optionally shorter) RNA strand (thin bar) to form a heteroduplex comprising a duplex portion and one or more (e.g., two) overhangs. In some embodiments, the second strand may comprise an RNA sequence complementary to a target sequence. For example, a second strand may be contacted with the first strand (or a library of first strands (each having the same or different sequences)) to form a duplex with the target sequence (if present) in a first strand. A method may further comprise contacting the heteroduplex with a dsDNase to form cleavage products. As illustrated, the dsDNase may cleave (digest) away the DNA strand of the duplex portion. In some embodiments, a first strand or a second strand may be linked to a surface (e.g., a bead, a plate, or a well), optionally through a linker.

FIG. 3D illustrates an example method comprising contacting a dsDNA comprising a top strand and a bottom strand (thick lines) and a ssRNA (optionally shorter than the dsDNA) to form a heteroduplex comprising the top strand of the dsDNA and the ssRNA. A heteroduplex may comprise a duplex portion and one or more (e.g., two) overhangs. As illustrated, the ssRNA (thin line) is linked to a surface by an optional linker. The bottom strand may be separated from the heteroduplex, for example, by washing the surface. A method may include contacting the heteroduplex with a dsDNase to form cleavage products. Cleavage products may include one or more fragments of the top strand (e.g., a 5′ fragment and a 3′ fragment).

FIG. 3E illustrates an example method comprising contacting a DNA strand (thick bar) with an RNA strand (thin bar) to form a heteroduplex comprising a duplex portion and one or more (e.g., two) overhangs. A method may further comprise cleaving overhangs (pictured) and/or purifying and/or analyzing the heteroduplex or any portion thereof. A method may further comprise dissociating (e.g., melting) the heteroduplex or any portion thereof to form separate strands and optionally analyzing (e.g., sequencing) or purifying one or more of the separated strands.

FIG. 4 shows an example of a method of using a dsDNase to reduce or remove repetitive DNA in a population of DNA molecules. A population of DNA molecules may be heated to separate the strands of duplex DNA and then cooled. DNA strands comprising repetitive sequences (thin lines) may reanneal more readily (e.g., with higher frequency) than DNA strands comprising more unique sequences (dashed and thick lines), which may tend to remain in single-stranded form. This resulting mixture of duplex and single-stranded DNA may be contacted with a dsDNase to digest the duplex (repetitive) DNA present while leaving intact the single-stranded (unique) DNA present.

FIG. 5 shows an example method of using a dsDNase to detect the presence of a target sequence in a population of RNA molecules. As shown, an RNA molecule (thin bar) comprising a target sequence (t) is contacted with a hairpin probe, the probe having a DNA loop comprising a sequence (t) complementary to the target (thick line) and an RNA stem comprising hybridized strands (thin lines), each strand comprising a complementary sequence (a or a′) to form a duplex comprising sequences t (RNA: thin line) and t′ (DNA: thick line). The duplex may be contacted with a dsDNase to produce cleavage products comprising the original RNA molecule comprising the target sequence and fragments a and a′ of the probe. A method may include signal amplification (upper dashed line) wherein the released RNA molecule contacts another hairpin probe molecule. Fragment a of the probe (short thin line) may contact a hairpin reporter comprising a DNA loop comprising the a sequence (thick line) and an RNA stem comprising strands (thin lines), each strand comprising a sequence complementary to the other (c and c′) with one strand further comprising a fluorophore (F) and the other strand further comprising a quencher (Q) to form a duplex comprising probe fragment a (RNA: thin line) and reporter loop a′ (DNA: thick line). The duplex may be contacted with a dsDNase to produce cleavage products comprising fragment a of the probe and fragments c and c′ of the reporter. A method may include signal amplification (lower dashed line) wherein the released probe fragment a contacts another hairpin reporter molecule. A fluorescent signal may result from separation of the fluorophore and the quencher (under appropriate illumination). In some embodiments, a probe may be immobilized on a surface (B) optionally via a linker.

FIG. 6 shows cleavage activity data for some example dsDNases on duplex DNA. P. pastoris strains expressing the indicated dsDNase gene under control of the GAP promoter were grown in BMGY media and culture supernatants used in dsDNase activity assays using a FAM-labeled 35-mer DNA hairpin probe. In this figure, an example P. monodon DNase is captioned “Pm”, an example C. opilio DNase is captioned “Co”, an example P. pelagicus DNase is captioned “Pp”, and an example A. chinensis DNase is captioned “Ac”.

FIG. 7 shows cleavage activity data for example dsDNases (DSN-2, DSN-1, and snow crab) on ssDNA and dsDNA substrates. Activity on a dsDNA substrate (35 nt dsDNA hairpin, open bars) or ssDNA substrate (15 nt ssDNA, black bars) was measured by monitoring the increase in fluorescence when the substrate is contacted with the indicated example dsDNase. All example enzymes tested here exhibit increased specificity for dsDNA over ssDNA (57 to 549-fold increased specificity for dsDNA compared to ssDNA).

FIG. 8 shows example data comparing the cleavage activity of some example dsDNases on duplex DNA in the absence and presence of calcium. dsDNase activity was measured by monitoring the increase in fluorescence when a quenched 35 nt hairpin dsDNA substrate is contacted with an example dsDNase in 1× Mg²⁺ Reaction Buffer (10 mM Tris-HCl, pH 7.9, 50 mM NaCl, 10 mM MgCl₂, 100 μg/ml Recombinant Albumin: open bars) or 1× Mg²⁺/Ca²⁺ Reaction Buffer (10 mM Tris-HCl, pH 7.6, 2.5 mM MgCl₂, 0.5 mM CaCl₂), black bars). All enzymes tested here exhibited increased activity in the presence of calcium.

FIG. 9) shows that activity of an example dsDNase (DSN-2) may decrease with increasing salt concentration. dsDNase activity was measured by monitoring the increase in fluorescence when a quenched 35 nt hairpin dsDNA substrate is contacted with DSN-2 in 1× Reaction Buffer (10 mM Tris-HCl, pH 7.6, 2.5 mM MgCl₂, 0.5 mM CaCl₂)) with increasing salt concentration (as indicated).

FIG. 10 shows example results of incubating a mixture of 20 pmol of dsDNA (60) bp oligonucleotide) and 20 pmol of ssDNA (50 nt oligonucleotide) with either DSN1 or DSN2 for 1 minute @25, 37, 50 or 65° C. Reaction products were run on a 4% E-gel EX gel (Invitrogen) stained with Sybr Gold. Lane (1) dsDNA PCR Marker, NEB #N3234, (2) SSDNA, (3) dsDNA (4) 25° ° C. (5) 37° C. (6) 50° C. (7) 65° C.

FIG. 11A shows an example of a fluorescent probe comprising a single-stranded 22 nt DNA oligo with a 5′ FAM label and a 3′ quencher (Black Hole Quencher-1). The short length of the oligo allows the quencher to quench the fluorophore. Cleavage of the ssDNA oligo releases FAM, resulting in an increase in fluorescence that can be measured at 517 nm using a spectrophotometer.

FIG. 11B shows example results of incubating a heteroduplex with a double-stranded nuclease (DSN-2) at 58° C. The heteroduplex was produced by hybridizing the 22 nt ssDNA probe shown in FIG. 11A with varying amounts of a Cluc mRNA (1763 nts). The Cluc mRNA was produced by in vitro transcription followed by DNase treatment to digest any remaining template DNA (prior to hybridization and prior to incubation with DSN2).

### BRIEF DESCRIPTION OF THE SEQUENCES

Some embodiments of this disclosure relate to the following provided sequences of example polynucleotides and/or example polypeptides.

SEQ ID NO: 1 is an example amino acid sequence of DSN-1, an example of a non-naturally occurring dsDNase.

SEQ ID NO: 2 is an example amino acid sequence of DSN-2, an example of a non-naturally occurring dsDNase.

SEQ ID NO: 3 is an example amino acid sequence of DSN-3, an example of a non-naturally occurring dsDNase.

SEQ ID NO: 4 is an example amino acid sequence of DSN-4, an example of a non-naturally occurring dsDNase.

SEQ ID NO: 5 is an example amino acid sequence of DSN-5, an example of a non-naturally occurring dsDNase.

SEQ ID NO: 6 is an example amino acid sequence of DSN-6, an example of a non-naturally occurring dsDNase.

SEQ ID NO: 7 is an example amino acid sequence of DSN-7, an example of a non-naturally occurring dsDNase.

SEQ ID NO: 8 is an example amino acid sequence of DSN-8, an example of a non-naturally occurring dsDNase.

SEQ ID NO: 9 is an example amino acid sequence of DSN-9, an example of a non-naturally occurring dsDNase.

SEQ ID NO: 10 is an example amino acid sequence of DSN-10, an example of a non-naturally occurring dsDNase.

SEQ ID NO: 11 is an example amino acid sequence of DSN-11, an example of a non-naturally occurring dsDNase.

SEQ ID NO: 12 is an example amino acid sequence of DSN-12, an example of a non-naturally occurring dsDNase.

SEQ ID NO: 13 is an example amino acid sequence of DSN-13, an example of a non-naturally occurring dsDNase.

SEQ ID NO: 14 is an example amino acid sequence of a snow crab (Chionoecetes opilio) DNase, an example dsDNase.

SEQ ID NO: 15 is an example amino acid sequence of a northern mauxia shrimp (Acetes chinensis) DNase, an example dsDNase.

SEQ ID NO: 16 is an example amino acid sequence of a northern mauxia shrimp (Acetes chinensis) DNase, an example dsDNase.

SEQ ID NO: 17 is an example amino acid sequence of a giant tiger prawn (Penaeus monodon) DNase, an example dsDNase.

SEQ ID NO: 18 is an example amino acid sequence of a blue swimming crab (Portunus pelagicus) DNase, an example dsDNase.

SEQ ID NO: 19 is an example sequence of a snow crab DNase variant, an example of a non-naturally occurring dsDNase. Amino acids 5-385 correspond to a DNase of Chionoecetes opilio.

SEQ ID NO: 20 is an example amino acid sequence of an N-terminal leader for use in protein expression and purification.

SEQ ID NO: 21 is an example amino acid sequence of a C-terminal linker and His6 tag for use (whether independently or together as exemplified in this sequence) in protein expression and purification.

SEQ ID NO: 22 is a DNA sequence of an example of a pD912casF amplification primer.

SEQ ID NO: 23 is a DNA sequence of an example of a pD912casR amplification primer.

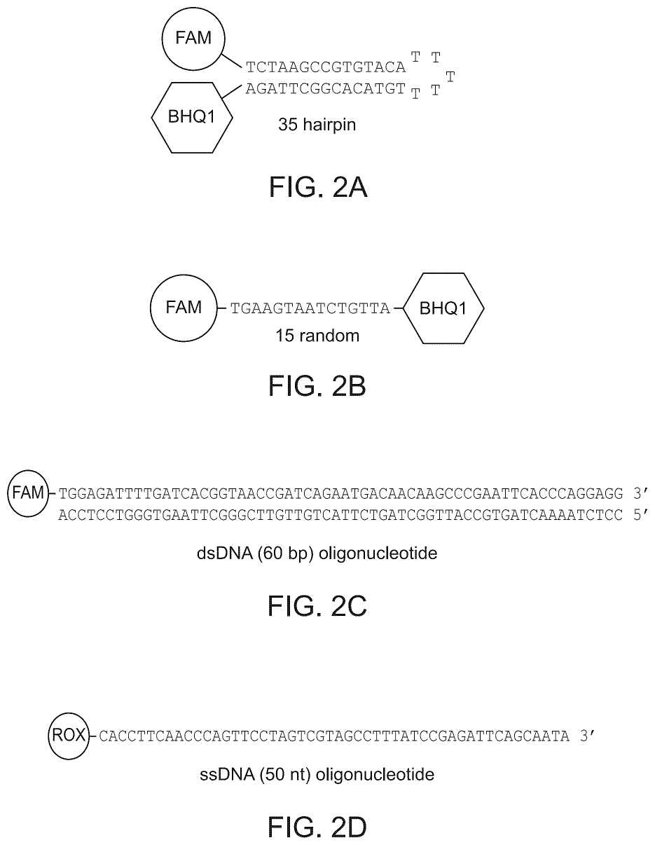

SEQ ID NO: 24 is a DNA sequence of an example of a 35 nt hairpin DNA oligo with a 5′ FAM label and a 3′ quencher shown in FIG. 2A.

SEQ ID NO: 25 is a DNA sequence of an example of a 15 nt single-stranded DNA oligo with a 5′ FAM label and a 3′ quencher shown in FIG. 2B.

SEQ ID NO: 26 is a DNA sequence of an example of a 60 nt linear dsDNA oligo with a 5′ FAM label shown in FIG. 2C.

SEQ ID NO: 27 is a DNA sequence of an example of a 50 nt single-stranded DNA oligo with a 5′ ROX label shown in FIG. 2D.

SEQ ID NO: 28 is an example amino acid sequence of DSN-1′, an example of a non-naturally occurring dsDNase.

SEQ ID NO: 29 is an example amino acid sequence of DSN-2′, an example of a non-naturally occurring dsDNase.

SEQ ID NO: 30 is an example nucleic acid sequence of Cluc mRNA used in Example 8.

SEQ ID NO:31 is an example amino acid sequence of DSN-CON1, an example of a non-naturally occurring dsDNase. Positions marked with “B” may be aspartate or asparagine. Positions marked with “J” may be leucine or isoleucine. Positions marked “X” may be any amino acid. Positions marked with “Z” may be glutamate or glutamine.

SEQ ID NO:32 is an example amino acid sequence of DSN-CON2, an example of a non-naturally occurring dsDNase. Positions marked “X” may be any amino acid. One or more positions of SEQ ID NO:32 may be identical to corresponding positions of SEQ ID NO:31.

### DETAILED DESCRIPTION

The present disclosure provides DNase variants, fusions, compositions, systems, apparatus, methods, and workflows for cleaving DNA. In some embodiments, DNase variants include dsDNases with desirable properties including, for example, selective digestion of duplex DNA and/or selective digestion of the DNA strand of a DNA/RNA hybrid.

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992): Lehninger, Biochemistry, Second Edition (Worth Publishers, New York, 1975); Strachan and Read, Human Molecular Genetics, Second Edition (Wiley-Liss, New York, 1999): Eckstein, editor, Oligonucleotides and Analogs: A Practical Approach (Oxford University Press, New York, 1991): Gait, editor, Oligonucleotide Synthesis: A Practical Approach (IRL Press, Oxford, 1984): Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample or example numerical values are provided, each alone may represent an intermediate value in a range of values and/or each may be combined with other sample or example values as the extremes of a range unless specified. For example, disclosures of values 42 and 53 in a figure and a value of 64 (with the same units) in a table may be intermediates in one or more ranges (e.g., 40-70, 35-75, and 30-80) and/or may be combined as endpoints of ranges 42-53, 42-64, and 53-64.

In the context of the present disclosure, “adapter” refers to a sequence that is joined to or can be joined to another molecule (e.g., ligated or copied onto via primer extension). An adapter can be DNA or RNA, or a mixture of the two. An adapter may be 15 to 100 bases, e.g., 50 to 70 bases, although adapters outside of this range are envisioned. In a library of polynucleotide molecules that contain an adapter (e.g., a 3′ or 5′ adapter, the adapter sequence used is not present in the DNA sequences under examination (i.e., the sequence in between the adapters). For example, if the library of polynucleotide molecules contains sequences derived from mammalian genomic DNA, cDNA or RNA, then the sequences of the adapters are not present in the mammalian genome under study. In many cases, the 5′ and 3′ adapters are of a different sequence and are not complementary. In many cases, an adapter will not contain a contiguous sequence of at least 8, 10 or 12 nucleotides that is found in the DNA under examination. Adapters may be designed to serve a specific purpose. For example, adapters may be designed for use in sequencing applications. Sequencing adapters may comprise, for example, an oligo-(dT) overhang, a barcode sequence, an overhang (other than oligo-(dT)) to anneal to another adapter, a site for anchoring a motor protein, and a sequence to bind to tethering oligos with affinity to polymer membrane for guiding a DNA or RNA fragment (on which it resides) to the vicinity of a nanopore, and combinations thereof.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine. A buffer composition may comprise a buffering agent and one or more additional components including, for example, salts (e.g., NaCl, MgCl₂), surfactants, detergents, stabilizers, and combinations thereof. Buffer compositions may be concentrated (e.g., 10×), dried, or lyophilized and subsequently diluted or wetted to a desired final working concentration. Buffer compositions include commercially available compositions (e.g., DNase I Reaction Buffer).

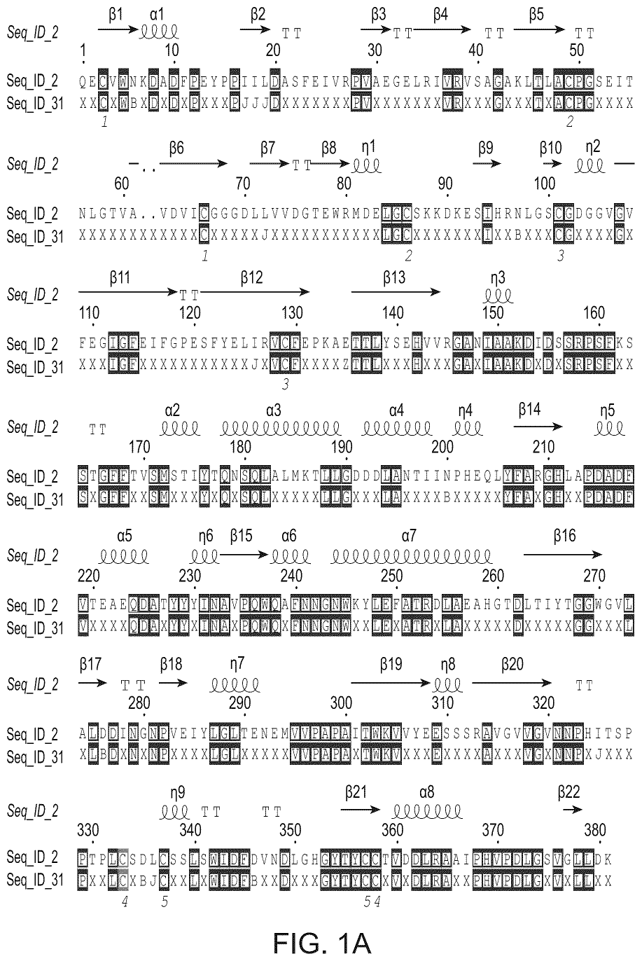

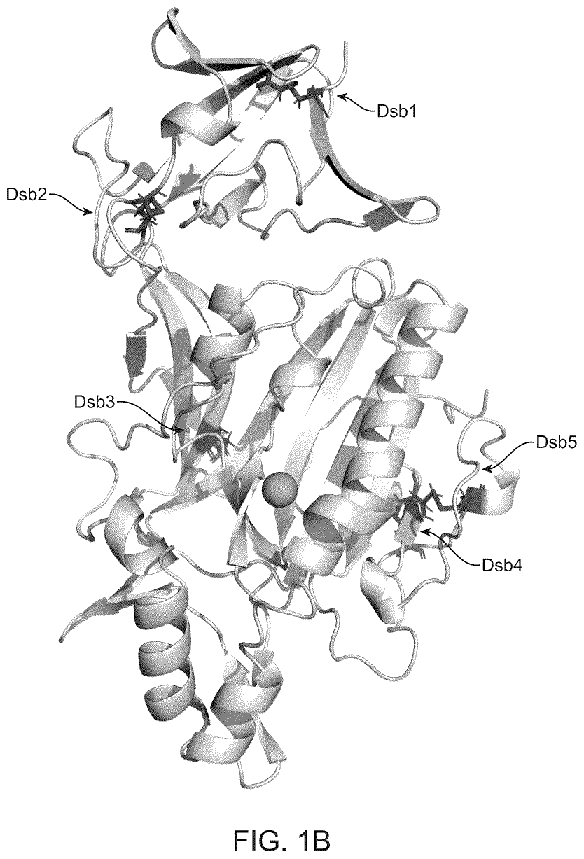

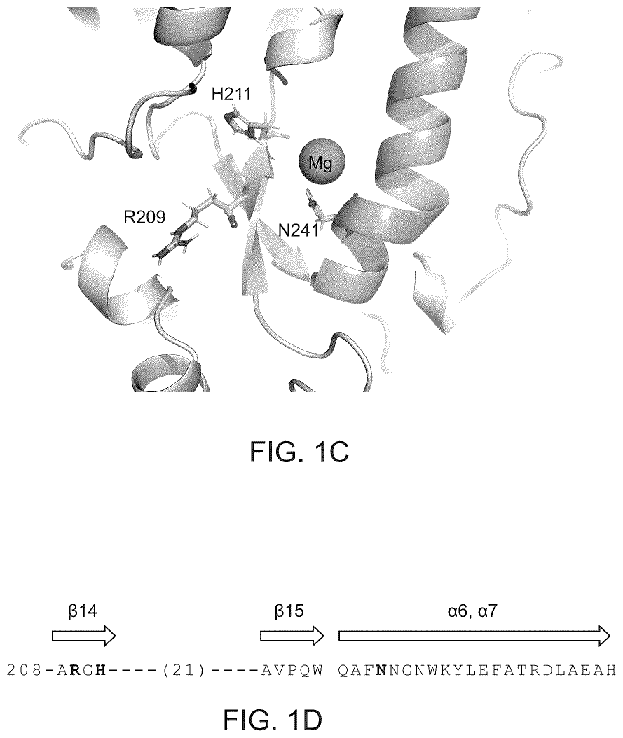

In the context of the present disclosure, “catalytically active” refers to the property of a molecule (e.g., a proteinaceous molecule or macromolecule) to function as a catalyst of one or more chemical reactions relative to one or more substrates and products. A catalytically active dsDNase, for example, hydrolyzes one or more polydeoxyribonucleic acids to yield (even if only briefly, for example, in the context of coupled reactions) products comprising at least one 5′-phosphorylated oligonucleotide or mononucleotide. Catalytic activity of dsDNases may be assessed using existing techniques applied to one or more model substrates (e.g., FIGS. 1A & 1B) and/or one or more substrates of interest. For example, effective assays for catalytic activity of dsDNases may include size fractionation of products (e.g., on gels or other matrices), radioactive assays, and fluorometric assays (e.g., #234056, #abab252898, AbCam PLC, Cambridge, U.K.: PicoGreen, Nucl. Acids Res. 2003 31(18):e111)). Catalytic activity may be assessed with respect to loss of original DNA (e.g., percent of original DNA remaining), concentration of DNA (e.g., DNA having a property of interest, for example, a size above 10 nts, above 8 nts, above 6 nts, above, above 3 nts in length, 4 nts, or above 2 nts in length), and/or metrics that serve as a proxy thereof.

Catalytic activity of a dsDNase may persist across a range of salt concentrations, temperatures and/or pH. For example, a dsDNase may display catalytic activity under such a range of conditions and/or following removal from exposure to conditions within such a range. A dsDNase may have catalytic activity at and/or following exposure to temperatures from 1° C. to 99° C., 10° C. to 90° C., 20° C. to 80° C., 4° C. to 37° C., 5° C. to 95° C., 10° C. to 30° C., 10° C. to 40° C., 10° ° C. to 50° C., 15° C. to 60° C., 15° C. to 45° C., 15° C. to 75° C., 50° C. to 99° C., 60° C. to 99° C., and/or 70° ° C. to 99° C. A dsDNase may have catalytic activity at and/or following exposure to a pH below 4, from 2 to 5, from 2 to 12, from 3 to 5, from 3 to 7, from 3 to 9, from 3 to 11, from 4 to 8, from 4 to 10, from 4 to 12, from 5 to 7, from 5 to 9, from 5 to 12, from 6 to 8, from 6 to 10, from 6 to 12, from 7 to 9, from 7 to 10, from 7 to 11, from 7 to 12, from 9 to 12, from 9 to 13, and/or over 11.

In the context of the present disclosure, with respect to polydeoxyribonucleic acids, “digest,” refers to hydrolyzing or otherwise reducing the size of such poly deoxyribonucleic acid. Unless qualified, digesting a polydeoxyribonucleic acid includes all degrees of hydrolyzing the phosphodiester backbone of or otherwise reducing the size of such polydeoxyribonucleic acid, partially up to and including fully reducing such polydeoxyribonucleotide to its constituent nucleotide or nucleoside monomers. Sites of hydrolysis may be regarded as independent of the nucleotide sequence (“non-specific”), even if some sequence bias is observed under some conditions.

In the context of the present disclosure, “dsDNase” refers to any naturally occurring enzyme or non-naturally occurring enzyme (e.g., an engineered enzyme) that hydrolyzes the phosphodiester backbone of one or more duplex poly deoxyribonucleic acid substrates to yield products comprising at least one 5′-phosphorylated polydeoxyribonucleotide and/or at least one 3′-hydroxylated polydeoxyribonucleotide. A dsDNase may display catalytic activity as a phosphodiesterase. Hydrolytic products of a polydeoxyribonucleotide contacted with a dsDNase may comprise DNA monomers, DNA oligomers (e.g., <10 nt), or both DNA monomers and DNA oligomers.

A dsDNase may comprise an amino acid sequence having at least 80%, at least 85%, at least 88% identical, at least 90%, at least 92%, at least 93%, at least 95%, at least 96%, at least 97%, at least 98% or at least 99% identity to any of SEQ ID NOS: 1-19, 28 or 29. A dsDNase having <100% identity to any of SEQ ID NOS: 1-19, 28 and 29 may have identical amino acids at any position within the given SEQ ID NO and/or may have non-identical amino acids at at any position within the given SEQ ID NO.

In some embodiments, one or more amino acids of any of SEQ ID NOS: 1-19, 28 and 29 may be conserved across catalytically active dsDNases. In some embodiments, identical amino acids may be found at consensus positions (e.g., active site residues). Non-identical amino acids may be found (e.g., may exclusively be found) at positions other than consensus positions. Examples of consensus sequences are provided at SEQ ID NO:31 and SEQ ID NO:32. Consensus positions of SEQ ID NO:31 are positions 3, 5, 6, 8, 10, 12, 16-20, 28, 29, 37, 38, 42, 46, 48-51, 68, 74, 86-88, 96, 99, 103, 104, 109, 114-116, 128, 130-132, 137-140, 144, 148, 149, 151-155, 157, 159-163, 166, 168-170, 173, 174, 178, 180, 182-184, 190-192, 196, 197, 202, 208-210, 212, 213, 216-221, 226-228, 230, 231, 233-235, 237-240, 242-247, 250, 252, 253-255, 257, 258, 264, 271, 272, 275, 277-279, 281, 283, 284, 298-291, 297-302, 311, 316, 320, 321, 323-325, 327, 331, 334, 335, 337-339, 342, 344-348, 351, 355-360, 362, 364-367, 370-376, 378, 380, and 381. Active site residues of SEQ ID NO:31 may include R211 (which, optionally, may be K211), H213, and/or N243. Consensus positions of SEQ ID NO:32 are positions 3, 5, 8, 10, 12, 16, 20, 28, 29, 37, 38, 42, 46, 48-51, 68, 86-88, 96, 103, 104, 109, 114-116, 130-132, 130-132, 138-140, 144, 148, 149, 151-155, 157, 159-163, 166, 168-170, 173, 174, 178, 180, 182-184, 190-192, 196, 197, 208-210, 212, 213, 216-221, 226-228, 230, 231, 233-235, 237-240, 242-247, 250, 252, 253-255, 257, 258, 264, 271, 272, 275, 277, 279, 281, 283, 284, 298-291, 297-302, 311, 316, 320, 321, 323-325, 331, 334, 335, 339, 342, 344-347, 351, 355-360, 362, 364-367, 370-376, 378, 380, and 381.

For example, a dsDNase having 379 amino acids and an amino acid sequence having 95% identity to SEQ ID NO: 1 is expected to have 360 amino acids identical to SEQ ID NO: 1 (0.95*379=360) and 19 non-identical amino acids (0.05*379=19). The 19 non-identical amino acids may be found outside of consensus positions (e.g., outside of the consensus positions of SEQ ID NO:31, outside of the consensus positions of SEQ ID NO:32). Thus, this example dsDNase may be described as having 95% identity to SEQ ID NO: 1 and 100% identity to SEQ ID NO:31 or may be described as having 95% identity to SEQ ID NO: 1 and 100% identity to SEQ ID NO:32.

For example, a dsDNase having an amino acid sequence having ≥80%, ≥85%, ≥88%, ≥90%, ≥92%, ≥93%, ≥95%, ≥96%, ≥97%, ≥98% or ≥99% identity to any of SEQ ID NOS: 1-19, 28 and 29 may further have (a) ≥99%, ≥99.2%, ≥99.4%, ≥99.6%, or 100% identity to SEQ ID NO:31 and/or (b) ≥99%, ≥99.2%, ≥99.4%, ≥99.6%, or 100% identity to SEQ ID NO:32. To illustrate, a dsDNase having an amino acid sequence with ≥80%, ≥85%, ≥88%, ≥90%, ≥92%, ≥93%, ≥95%, ≥96%, ≥97%, ≥98% or ≥99% identity to any of SEQ ID NOS: 1-19, 28 or 29 may have ≥99%, ≥99.2%, ≥99.4%, ≥99.6%, or 100% identity to SEQ ID NO:31 at its positions corresponding to positions 3, 5, 6, 8, 10, 12, 16-20, 28, 29, 37, 38, 42, 46, 48-51, 68, 74, 86-88, 96, 99, 103, 104, 109, 114-116, 128, 130-132, 137-140, 144, 148, 149, 151-155, 157, 159-163, 166, 168-170, 173, 174, 178, 180, 182-184, 190-192, 196, 197, 202, 208-210, 212, 213, 216-221, 226-228, 230, 231, 233-235, 237-240, 242-247, 250, 252, 253-255, 257, 258, 264, 271, 272, 275, 277-279, 281, 283, 284, 298-291, 297-302, 311, 316, 320, 321, 323-325, 327, 331, 334, 335, 337-339, 342, 344-348, 351, 355-360, 362, 364-367, 370-376, 378, 380, 381 of SEQ ID NO: 31. To further illustrate, a dsDNase having an amino acid sequence with ≥80%, ≥85%, ≥88%, ≥90%, ≥92%, ≥93%, ≥95%, ≥96%, ≥97%, ≥98% or ≥99% identity to any of SEQ ID NOS: 1-19, 28 or 29 may have ≥99%, ≥99.2%, ≥ 99.4%, ≥99.6%, or 100% identity with SEQ ID NO:32 at its positions corresponding to positions 3, 5, 8, 10, 12, 16, 20, 28, 29, 37, 38, 42, 46, 48-51, 68, 86-88, 96, 103, 104, 109, 114-116, 130-132, 130-132, 138-140, 144, 148, 149, 151-155, 157, 159-163, 166, 168-170, 173, 174, 178, 180, 182-184, 190-192, 196, 197, 208-210, 212, 213, 216-221, 226-228, 230, 231, 233-235, 237-240, 242-247, 250, 252, 253-255, 257, 258, 264, 271, 272, 275, 277, 279, 281, 283, 284, 298-291, 297-302, 311, 316, 320, 321, 323-325, 331, 334, 335, 339, 342, 344-347, 351, 355-360, 362, 364-367, 370-376, 378, 380, 381 of SEQ ID NO: 32. As these illustrations demonstrate, a limited degree of variability may be tolerated at otherwise conserved positions.

In some embodiments, a dsDNase having an amino acid sequence with ≥80%, ≥85%, ≥88%, ≥90%, ≥92%, ≥93%, ≥95%, ≥96%, ≥97%, ≥98% or ≥99% identity to SEQ ID NO:2 may be identical to SEQ ID NO:31 at its position corresponding to position 216 (proline) of SEQ ID NO:31 and, optionally, may be as thermotolerant as a sequence that is 100% identical to SEQ ID NO:2. Unless a thermolabile dsDNase is desired, any dsDNase of the disclosure may have/retain this conserved proline residue at a position corresponding to position 216 of SEQ ID NO:31. In some embodiments, a dsDNase having an amino acid sequence with ≥80%, ≥85%, ≥88%, ≥90%, ≥92%, ≥93%, ≥95%, ≥96%, ≥97%, ≥98% or ≥99% identity to SEQ ID NO:2 (or optionally any of SEQ ID NO: 1, 3-19, 28, or 29) may be identical to SEQ ID NO:31 at its position(s) corresponding to position 209 (arginine or optionally lysine), 211 (histidine), and/or 241 (asparagine) of SEQ ID NO:2. In some embodiments, a dsDNase having an amino acid sequence with ≥80%, ≥85%, ≥88%, ≥90%, ≥92%, ≥93%, ≥95%, ≥96%, ≥97%, ≥98% or ≥99% identity to SEQ ID NO:2 (or optionally any of SEQ ID NO:1, 3-19, 28, or 29) may be identical to SEQ ID NO:31 and may comprise (i) a cysteine residue at its position corresponding to position 3 (C3) of SEQ ID NO:2, (ii) a cysteine residue at its position corresponding to position 49 (C49) of SEQ ID NO:2, (iii) a threonine residue at its position corresponding to position 55 (T55) of SEQ ID NO:2, (iv) a cysteine residue at its position corresponding to position 66 (C66) of SEQ ID NO:2, (v) a cysteine residue at its position corresponding to position 86 (C86) of SEQ ID NO:2, (vi) a serine residue at its position corresponding to position 87 (S87) of SEQ ID NO:2, (vii) a lysine residue at its position corresponding to position 88 (K88) of SEQ ID NO:2, (viii) a cysteine residue at its position corresponding to position 101 (C101) of SEQ ID NO:2, (ix) a cysteine residue at its position corresponding to position 129 (C129) of SEQ ID NO:2, (x) an arginine residue at its position corresponding to position 158 (R158) of SEQ ID NO:2, (xi) an isoleucine residue at its position corresponding to position 175 (1175) of SEQ ID NO:2, (xii) an arginine residue at its position corresponding to position 209 (R209) of SEQ ID NO:2, (xiii) a histidine residue at its position corresponding to position 211 (H211) of SEQ ID NO:2, (xiv) a proline residue at its position corresponding to position 216 (P216) of SEQ ID NO:2, (xv) an asparagine residue at its position corresponding to position 241 (N241) of SEQ ID NO:2, (xvi) a tryptophane at its position corresponding to position 270 (W270) of SEQ ID NO:2, (xvii) a glycine at its position corresponding to position 280 (G280) of SEQ ID NO:2, (xviii) a cysteine residue at its position corresponding to position 333 (C333) of SEQ ID NO:2, (xix) a cysteine residue at its position corresponding to position 337 (C337) of SEQ ID NO:2, (xx) a cysteine residue at its position corresponding to position 357 (C357) of SEQ ID NO:2, and/or (xxi) a cysteine residue at its position corresponding to position 358 (C358) of SEQ ID NO:2. dsDNase (e.g., a selective dsDNase), according to some embodiments, may have a secondary structure according to FIG. 1A and/or a tertiary and/or quaternary structure according to FIGS. 1B and 1C. For example, a dsDNase (e.g., a selective dsDNase) may have an N-terminal cap domain (residues 1 to 88 of SEQ ID NO:2) and a catalytic nuclease (NUC) domain (residues 89 to 381 of SEQ ID NO:2), according to some embodiments. The NUC domain includes a ββα-metal motif typical of NUC enzymes, shown in FIGS. 1C and 1D. Within this ββα-metal motif, asparagine 241 coordinates a Mg ion that is essential for enzyme activity. Histidine 211 acts as a general base and may be an essential catalytic residue. The nearby residue arginine 209 is also important for activity and may be either an arginine or lysine.

A dsDNase may comprise, for example, an amino acid sequence having ≥99%, ≥99.2%, ≥99.4%, ≥99.6%, or 100% identity to SEQ ID NO:31, its position corresponding to position 211 of SEQ ID NO:31 is arginine or lysine, its position corresponding to position 213 of SEQ ID NO:31 is histidine (H213), and its position corresponding to position 243 of SEQ ID NO:31 is asparagine (N243), and wherein R211 (or K211), H213, and N243 are in catalytic communication with (a) one another, (b) a Mg ion, (c) a DNA strand of a DNA:RNA duplex, and/or (d) a DNA strand of a DNA:DNA duplex. In this context, amino acids may be said to be in catalytic communication when they are in sufficient physical and chemical proximity to confer or support catalytic activity of the dsDNase. A dsDNase (e.g., a selective dsDNase), according to some embodiments, may comprise an amino acid sequence having ≥99%, 99.2%, ≥99.4%, ≥99.6%, or 100% identity to SEQ ID NO:31 and further comprise an active site comprising (or, optionally, defined by) an arginine or lysine at its position corresponding to position 211 of SEQ ID NO:31 (R211), a histidine at its position corresponding to position 213 of SEQ ID NO:31 (H213), and an asparagine at its position corresponding to position 243 of SEQ ID NO:31 (N243). In some embodiments, a dsDNase (e.g., a selective dsDNase) may comprise an amino acid sequence having ≥99%, ≥99.2%, ≥99.4%, ≥99.6%, or 100% identity to SEQ ID NO:31, wherein its positions corresponding to positions 3, 49, 68, 88, 103, 131, 335, 339, 359, and 360 of SEQ ID NO:31 are each cysteine residues (C3, C49, C68, C88, C103, C131, C335, C339, C359, and C360) and wherein the dsDNase may have a 3D structure that includes up to 5 disulfide bonds, the disulfide bonds selected from C3-C68, C49-C88, C103-C131, C335-C360, and C339-C359 (FIG. 1B). An optional sixth disulfide bond may occur at C315-379. While a dsDNase most typically has 5 or 6 disulfide bonds, fewer or more disulfide bonds are possible. A dsDNase (e.g., a selective dsDNase) may comprise an amino acid sequence having ≥99%, ≥99.2%, ≥99.4%, ≥99.6%, or 100% identity to SEQ ID NO:31, wherein its position corresponding to position 55 of SEQ ID NO:31 is threonine (T55), its position corresponding to position 177 of SEQ ID NO:31 is isoleucine (1177), or both its position corresponding to position 55 of SEQ ID NO:31 is threonine (T55) and its position corresponding to position 177 of SEQ ID NO:31 is isoleucine (1177). A dsDNase (e.g., a selective dsDNase) may comprise an amino acid sequence having ≥99%, ≥99.2%, ≥99.4%, ≥99.6%, or 100% identity to SEQ ID NO:31, wherein its position corresponding to position 55 of SEQ ID NO:31 is threonine (T55), its position corresponding to position 89 of SEQ ID NO:31 is serine (S89), its position corresponding to position 90 of SEQ ID NO:31 is lysine (K90), its position corresponding to position 177 of SEQ ID NO:31 is isoleucine (1177), its position corresponding to position 211 of SEQ ID NO:31 is lysine or arginine (K211 or R211), its position corresponding to position 272 of SEQ ID NO:31 is tryptophane (W272), and/or its position corresponding to position 282 of SEQ ID NO:31 is glycine (G282). A dsDNase (e.g., a selective dsDNase) may comprise an amino acid sequence having ≥99%, ≥99.2%, ≥99.4%, ≥99.6%, or 100% identity to SEQ ID NO:31, wherein its position corresponding to position 3 of SEQ ID NO:31 is cysteine (C3), its position corresponding to position 49 of SEQ ID NO:31 is cysteine (C49), its position corresponding to position 55 of SEQ ID NO:31 is threonine (T55), its position corresponding to position 68 of SEQ ID NO:31 is cysteine (C68), its position corresponding to position 88 of SEQ ID NO:31 is cysteine (C88), its position corresponding to position 89 of SEQ ID NO:31 is serine (S89), its position corresponding to position 90 of SEQ ID NO:31 is lysine (K90), its position corresponding to position 103 of SEQ ID NO:31 is cysteine (C103), its position corresponding to position 131 of SEQ ID NO:31 is cysteine (C131), its position corresponding to position 177 of SEQ ID NO:31 is isoleucine (1177), its position corresponding to position 211 of SEQ ID NO:31 is lysine or arginine (K211 or R211), its position corresponding to position 213 of SEQ ID NO:31 is histidine (H213), its position corresponding to position 243 of SEQ ID NO:31 is asparagine (N243), its position corresponding to position 272 of SEQ ID NO:31 is tryptophane (W272), its position corresponding to position 282 of SEQ ID NO:31 is glycine (G282), its position corresponding to position 335 of SEQ ID NO:31 is cysteine (C335), its position corresponding to position 339 of SEQ ID NO:31 is cysteine (C339), its position corresponding to position 359 of SEQ ID NO:31 is cysteine (C359), and/or its position corresponding to position 360 of SEQ ID NO:31 is cysteine (C360), A dsDNase (e.g., as set forth in this paragraph) may comprise an amino acid sequence with ≥80%, ≥85%, ≥88%, ≥90%, ≥92%, ≥93%, ≥95%, ≥96%, ≥97%, ≥98% or ≥99% identity to any of SEQ ID NO:1-19, 28, or 29) in addition to it's similarity/identity to SEQ ID NO:31.

dsDNases include fusion proteins comprising (a) a first polypeptide having at least 80%, at least 85%, at least 90%, at least 93%, at least 96%, at least 97%, at least 98% or at least 99% identity to any of SEQ ID NOS: 1-18 and (b) a second polypeptide (e.g., albumin, a DNA-binding domain, a topoisomerase, a secretion signal, a linker and/or a purification tag). Example fusions include SEQ ID NO: 19, 28 and 29), wherein (a) may be N-terminal to (b) or (b) may be N-terminal to (a). If (b) is a linker, a dsDNase may further comprise (c) a third polypeptide (e.g., a albumin, a DNA-binding domain, a topoisomerase, a secretion signal, and/or a purification tag). dsDNases include fusions comprising (a) a first polypeptide having at least 80%, at least 85%, at least 90%, at least 93%, at least 96%, at least 97%, at least 98% or at least 99% identity to any of SEQ ID NO: 1-18 and (b) one or more additional materials, examples of which include, without limitation, a carbohydrate (e.g., a glycoside), a fatty acid, a surface (e.g., a bead, a plate, or a well), a purification tag (e.g., a histidine tag), a label, and other molecules (e.g., polyethylene glycol). Fusions may be covalently attached at the N-terminal end of the polypeptide, the C-terminal end of the polypeptide, or at an amino acid position (e.g., an amino acid side chain) along the length of the polypeptide. A dsDNase may comprise one or more modified amino acids (e.g., γ-carboxyglutamic acid, 4-hydroxyproline, 5-hydroxylysine, and selenocysteine) and/or D-amino acids.

A dsDNase may be active in the absence of magnesium. A dsDNase may be active in the presence of magnesium (e.g., ≤1 mM MgCl₂, ≤2.5 mM MgCl₂, ≤5 mM MgCl₂, ≤10 mM MgCl₂, ≤15 mM MgCl₂, ≤20 mM MgCl₂, ≤25 mM MgCl₂, ≤35 mM MgCl₂, ≤50 mM MgCl₂, in each case, inclusive, or over ≥50 mM MgCl₂). A dsDNase may bind magnesium cations and such magnesium cations may support or participate in DNA cleavage. In some embodiments, a dsDNase may have one Mg²⁺ binding site, two Mg²⁺ binding sites, or more than two Mg²⁺ binding sites. The Mg²⁺ binding affinity of a dsDNase may be similar or different than the binding affinity of a DNase I, according to some embodiments. For example, the Mg²⁺ binding affinity of a dsDNase may be in a range of 0.25×10⁻⁴M, 0.5×10⁻⁴M, 0.75×10⁻⁴M, or 1.0×10⁻⁴M to 2.5×10⁻⁴M, 5×10⁻⁴M, 7.5×10⁻⁴M, 10×10⁻⁴M, or 25×10⁻⁴M (e.g., 0.25×10⁻⁴M to 2.5×10⁻⁴M or 0.5×10⁻⁴M to 5×10⁻⁴M or 0.75×10⁻⁴M to 7.5×10⁻⁴M).

A dsDNase may be active in the absence of calcium. A dsDNase may be active in the presence of calcium (e.g., ≤100 μM CaCl₂), ≤250 μM CaCl₂), ≤500 μM CaCl₂), ≤750 μM CaCl₂, ≤1 mM CaCl₂), ≤2.5 mM CaCl₂), ≤5 mM CaCl₂), ≤10 mM CaCl₂), in each case, inclusive, or over ≥10 mM CaCl₂). Activity of a dsDNase may increase with increasing concentration of calcium (e.g., over a range of 0.1 μM to 20 mM). A dsDNase may bind calcium cations and such calcium cations may support or participate in DNA cleavage. In some embodiments, a dsDNase may have one Ca²⁺ binding site, two Ca²⁺ binding sites, or more than two Ca²⁺ binding sites. The Ca²⁺ binding affinity of a dsDNase may be similar or different than the binding affinity of a DNase I, according to some embodiments. For example, the Ca²⁺ binding affinity of a dsDNase may be in a range of 0.15×10⁻⁵M, 0.5×10⁻⁵M, 0.75×10⁻⁵M, or 1.0×10⁻⁵M to 2.0×10⁻⁵M, 5×10⁻⁵M, 7.5×10⁻⁵M, 10×10⁻⁵M, or 20×10⁻⁵M (e.g., 0.15×10⁻⁵M to 2.0×10⁻⁵M or 0.5×10⁻⁵M to 5×10⁻⁵M or 0.75×10⁻⁵M to 7.5×10⁻⁵M).

Activity of a dsDNase may be higher (e.g., 2-fold higher, 3-fold higher, 4-fold higher, 5-fold higher, 6-fold higher, 7-fold higher, 8-fold higher, 9-fold higher, 10-fold higher, 11-fold higher, 12-fold higher, 13-fold higher, 14-fold higher, 15-fold higher) in the presence of both calcium (e.g., at least 100 μM CaCl₂), at least 250 μM CaCl₂), at least 500 μM CaCl₂)) and magnesium (e.g., at least 1 mM MgCl₂, at least 2.5 mM MgCl₂, at least 10 mM MgCl₂) than its activity in the presence of calcium without magnesium or its activity in the presence of magnesium without calcium.

A dsDNase may be soluble in aqueous solutions free of surfactants and/or free of detergents. A dsDNase may have a structure in a bacterial cell and/or a yeast cell that is catalytically active. For example, microbially (e.g., E. coli, Pichia) expressed wild type dsDNase from Penaeus japonicus (Wang et al., Biochem. J., 2000, 346:799-804) may have a structure that is catalytically in active while a variant of wild type dsDNase from Penaeus japonicus (e.g., SEQ ID NO:2) may have a structure that is active in the host and such activity may persist upon purification from the host cells.

Catalytic activity of a dsDNA may be denominated in units, wherein one unit is the amount of enzyme needed to release 50 pmol of FAM from a 35mer FAM-BHQ1 labeled hairpin oligonucleotide in 1 minute at 30° C. in a 50 μl reaction volume in 1× NEBuffer r1.1 (10 mM Bis-Tris-Propane-HCl, 10 mM MgCl₂, 100 μg/ml recombinant albumin, pH 7.0) @25° ° C.

A dsDNase may be said to be “selective”, “specific” or have “selective activity” with respect to a property of cleaving one material (e.g., a substrate) more effectively, more efficiently, more quickly, more completely, or otherwise to a greater extent than another material. For example, a selective dsDNase may produce more cleavage events (e.g., ≥2-fold, ≥3-fold, ≥5-fold, ≥10-fold, ≥20-fold, ≥25-fold, ≥50-fold, ≥100-fold, ≥200-fold, ≥250-fold, ≥500-fold, ≥1000-fold more cleavage events) when the enzyme is contacted with duplex polynucleotides comprising DNA (e.g., DNA:DNA or DNA:RNA) than when contacted with single-stranded DNA, single-stranded RNA, and/or duplex RNA under otherwise identical conditions (e.g., time, reaction buffer composition, reaction pH, salt(s) present, reaction temperature). In some embodiments, selectivity may vary with one or more reaction conditions. In some embodiments, a dsDNase may have selective activity at substrate to enzyme ratios (e.g., mass ratios, molar ratios) of 10, 50, or 100 to 100, 500, or 1,000. A selective dsDNase, according to some embodiments, may cleave DNA strands of a DNA:RNA duplex more frequently than the RNA strands. According to some embodiments, a selective dsDNase may cleave ≥75% (or ≥80% or ≥85% or ≥90% or ≥95% or ≥98%) of the DNA strands of a DNA:RNA duplex at least once and cleave ≤25% (or ≤20% or ≤15% or ≤10% or ≤5% or ≤2%) of the RNA strands even once, for example, where the DNA:RNA duplex has ≤20 mismatches (or ≤15 mismatches or ≤10 mismatches or ≤5 mismatches or ≤2 mismatches) per 100 nucleotides (or per 1,000 nucleotides). For example, a selective dsDNase may cleave ≥95% of the DNA strands of a DNA:RNA duplex at least once and cleave ≤5% of the RNA strands even once, wherein the DNA:RNA duplex is ≤3 kb and has no mismatched bases. In the context of the present disclosure, “duplex” and “double stranded” refer to any conformation of a polynucleotide in which two polynucleotide strands (e.g., separate molecules or spatially separated portions of a single molecule) are arranged anti parallel to one another in a helix (e.g., A-form, B-form, Z-form) with complementary bases of each strand paired with one another (e.g., in Watson-Crick base pairs). Paired bases may be stacked relative to one another to permit pi electrons of the bases to be shared. A polynucleotide may have a homoduplex conformation (e.g., DNA:DNA or RNA:RNA) or a heteroduplex confirmation (e.g., DNA:RNA).

Duplex stability, in part, may be related to the ratio of complementary bases to mismatches (if any) in the two strands, ratio of pairs with three hydrogen bonds (e.g., G:C) to pairs with two hydrogen bonds (e.g., A:T, A:U) in the duplex, and the length of the strands with higher ratios and longer strands generally associated with higher stability. Duplex stability, in part, may be related to ambient conditions including, for example, temperature, pH, salinity, and/or the presence, concentration and identity of any buffer(s), denaturant(s) (e.g., formamide), crowding agent(s) (e.g., PEG), detergent(s) (e.g., SDS), surfactant(s), polysaccharide(s) (e.g., dextran sulfate), chelator(s) (e.g., EDTA), and nucleic acid(s) (e.g., salmon sperm DNA). A duplex polynucleotide may comprise one or more unpaired bases including, for example, a mismatched base, a hairpin loop, a single-stranded (5′ and/or 3′) end.

Duplex polynucleotides (e.g., dsDNase substrates) may have any desired length and/or any desired number of mismatched bases. For example, a duplex polynucleotide may have a length of ≤10 nucleotides, 10-25 nucleotides, 10-200 nucleotides, ≤50 nucleotides, 50-500 nucleotides, 50-2000 nucleotides, ≤2 kb, and/or 2-10 kb. Duplex polynucleotides may have any desired number of mismatched nucleotides, for example, over 90%, over 95%, over 97%, over 98 and/or over 99% sequence identity. Duplex polynucleotides (e.g., dsDNase substrates) may have no more than one mismatch per 10 nucleotides, no more than 1 mismatch per 100 nucleotides, no more than 2 mismatches per 100 nucleotides, and/or no more than 3 mismatches per 100 nucleotides.

In the context of the present disclosure, “engineered dsDNase” refers to any non-naturally occurring dsDNase. An engineered dsDNase may have any number of insertions, deletions, or substitutions relative to a naturally occurring enzyme. For example, an engineered dsDNase may have less than 100% identity, less than 99% identity, less than 98% identity, less than 90% identity, less than 85% identity, less than 80% identity, less than 70% identity, less than 60% identity, less than 50% identity, less than 40% identity, less than 30% identity, or less than 20% identity to a naturally occurring enzyme. Engineered dsDNases may include expression tags and/or purification tags. Examples of engineered dsDNases include SEQ ID NOS: 1-13. Examples of engineered dsDNases also include variants of SEQ ID NOS: 14-18, including variants having expression and/or purification tags illustrated, for example, by SEQ ID NO: 19.

In the context of the present disclosure. “fusion protein” refers to a protein composed of two or more polypeptide components that are un-joined in their native state. Fusion proteins may be a combination of two, three or four or more different proteins. For example, a fusion protein may comprise two naturally occurring polypeptides that are not joined in their respective native states. A fusion protein may comprise two polypeptides, one of which is naturally occurring and the other of which is non-naturally occurring. The term polypeptide is not intended to be limited to a fusion of two heterologous amino acid sequences. A fusion protein may have one or more heterologous domains added to the N-terminus, C-terminus, and or the middle portion of the protein. If two parts of a fusion protein are “heterologous”, they are not part of the same protein in its natural state. Examples of fusion proteins include a dsDNase fused to an SSO7d DNA binding peptide (see for example, U.S. Pat. No. 6,627,424), a transcription factor (see for example, U.S. Pat. No. 10,041,051), an antibody, protein A (e.g., SpA), a binding domain suitable for immobilization such as maltose binding domain (MBP), a histidine tag (“His-tag”: e.g., SEQ ID NOS: 19, 21, 28, & 29), chitin binding domain), alpha mating factor (SEQ ID NOS: 19-20, 28 & 29) or a SNAP-Tag® (New England Biolabs, Ipswich, MA (see for example U.S. Pat. Nos. 7,939,284 and 7,888,090)), and/or albumin. The binding peptide may be used to improve solubility or yield of the dsDNase during the production of the protein reagent. Other examples of fusion proteins include fusions of a DNase I and a heterologous targeting sequence, a linker, an epitope tag, a detectable fusion partner, such as a fluorescent protein, ß-galactosidase, luciferase and/or functionally similar peptides. Components of a fusion protein may be joined by one or more peptide bonds, disulfide linkages, and/or other covalent bonds.

In the context of the present disclosure, “identity” or “identical”, in the context of amino acids or nucleotides, refers to the presence of the same amino acid or the same nucleobase at a given position in a polypeptide or polynucleotide, respectively. For clarity, if a reference sequence designates an “Asx” or “B” at a given position, a query sequence is said to be identical to the reference sequence if the query sequence has an aspartate or an asparagine at its position corresponding to the given reference sequence position: if a reference sequence designates an “Glx” or “Z” at a given position, a query sequence is said to be identical to the reference sequence if the query sequence has a glutamate or a glutamine at its position corresponding to the given reference sequence position; and/or if a reference sequence designates an “Xle” or “J” at a given position, a query sequence is said to be identical to the reference sequence if the query sequence has a leucine or an isoleucine at its position corresponding to the given reference sequence position.

In the context of the present disclosure, “library” or “polynucleotide library” refers to a mixture of different molecules. A library may comprise DNA and/or RNA (e.g., genomic DNA, organelle DNA, cDNA, mRNA, microRNA, long non-coding RNAs or other RNAs of interest) or fragments thereof from any desired source (e.g., human, non-human mammal, plant, microbe, virus, or synthetic). A library may have any desired number of different polynucleotides. For example, a library may have more than 10⁴, 10⁵, 10⁶ or 10⁷ different nucleic acid molecules. A library may have fewer different molecules, for example, where the molecules collectively have more than 10⁴, 10⁵, 10⁶ or 10⁷ or more nucleotides. In some embodiments, a library of polynucleotide molecules may be an enriched library, in which case the library may have a complexity of less than 10%, less than 5%, less than 1%, less than 0.5%, or less than 0.1%, less than 0.01%, less than 0.001% or less than 0.0001% relative to the unenriched sample (e.g., a sample made from total RNA or total genomic DNA from a eukaryotic cell sample. Molecules can be enriched by methods such as described in US2014/0287468 or US 2015/0119261. A library, in some embodiments, may include member polynucleotides that are tagged with an adapter.

In the context of the present disclosure, “magnetic bead” refers to a support comprising a surface and a core. A surface may comprise one or more surface modifications including polyethylene glycol (e.g., O⁶-benzyleguanine and/or PEG₇₅₀). A magnetic core may comprise one or more magnetic or magnetizable materials including, for example, iron, an iron oxide, cobalt a cobalt oxide, nickel, a nickel oxide, or combinations thereof.

In the context of the present disclosure, “magnetically gathering” refers to subjecting a material and/or fluid (e.g., a dsDNase reaction mixture on a surface or in a container) to a magnetic field to spatially gather or concentrate any components of the material and/or fluid comprising a ferromagnetic metal (e.g., iron, nickel, cobalt). Subjecting a material and/or fluid to a magnetic field may be accomplished in any manner desired. For example, a material and/or fluid may be moved into an existing magnetic field, an existing magnet may be moved into effective range of a material and/or fluid, or a magnetic field may be applied (e.g., by reshaping a magnetic field or by switching on an electromagnet) within an effective distance of a material and/or fluid. In some embodiments, subjecting a fluid reaction mixture comprising magnetic beads to a magnetic field gathers the magnetic beads in the reaction forming a fluid fraction (supernatant) comprising, for example, reaction products, buffers, and solvent, but few, if any, magnetic beads) and a bead fraction comprising, for example, magnetic beads (and linked polynucleotides), but few, if any, molecules of enzymes (e.g., dsDNase) or free polynucleotides. Magnetically gathering magnetic beads (and linked polynucleotides) may include forming a pellet or other aggregate of magnetic beads. Such pellet or other aggregate may be sufficiently well formed and stable to tolerate manipulation or removal of a fluid fraction (e.g., supernatant) adjoining and/or in contact with the pellet or other aggregate.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides, polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature. (c) omitting one or components otherwise found in naturally occurring compositions. (d) having a form not found in nature, e.g., dried, freeze dried, crystalline, aqueous, and (e) having one or more additional components beyond those found in nature (e.g., buffering agents, a detergent, a dye, a solvent or a preservative).

With reference to an amino acid, “position” refers to the place such amino acid occupies in the primary sequence of a peptide or polypeptide numbered from its amino terminus to its carboxy terminus. A position in one primary sequence may correspond to a position in a second primary sequence, for example, where the two positions are opposite one another when the two primary sequences are aligned using an alignment algorithm (e.g., BLAST (Journal of Molecular Biology. 215 (3): 403-410) using default parameters (e.g., expect threshold 0.05, word size 3, max matches in a query range 0), matrix BLOSUM62, Gap existence 11 extension 1, and conditional compositional score matrix adjustment) or custom parameters). An amino acid position in one sequence may correspond to a position within a functionally equivalent motif or structural motif that can be identified within one or more other sequence(s) in a database by alignment of the motifs.

In the context of the present disclosure, “reporter” refers to a molecule comprising a single-stranded DNA having ends, each comprising a terminal nucleotide (e.g., a 5′ nucleotide or a 3′ nucleotide). A reporter further may include a signal moiety comprising, for example, a fluorophore, a phosphor, an isotope, a quantum dot, and/or a nanoparticle. A reporter comprising a fluorophore optionally may further comprise a quencher corresponding to the fluorophore. A quencher may be regarded to correspond to a fluorophore where the fluorophore, in proximity to the quencher, emits little or no light despite being illuminated by one or more of its excitation frequencies. A signal moiety and/or a quencher may be joined to the DNA strand, for example, through a modified nucleotide positioned at the 5′ end, the 3″ end, or anywhere between. A single-stranded DNA may have any desired length and/or sequence. For example, a single-stranded DNA of a reporter may have a length of 10 nucleotides or less, 12 nucleotides or less, 14 nucleotides or less, 16 nucleotides or less, 18 nucleotides or less, 20) nucleotides or less, 30 nucleotides or less, 40 nucleotides or less or 50 nucleotides or less. A DNA sequence of a reporter may have, for example, a sequence configured to resist formation of intramolecular duplexes (e.g., dG₁₅, dT₁₅). A DNA sequence of a reporter may have, for example, a sequence configured to form an intramolecular duplex (e.g., having two complementary portions to form a stem with an interconnecting loop).

As used herein, “salt tolerant” refers to a property or capacity to display activity in the presence of one or more salts. A salt tolerant dsDNase, for example, may display DNA-binding activity and/or catalytic activity in the presence of one or more salts. Binding activity may be evaluated in suitable biochemical terms, for example, binding affinity (Kd). Similarly, catalytic activity may be evaluated in suitable biochemical terms, for example, Michaelis constant (Km) and/or maximal reaction velocity (Vmax). Catalytic activity and/or DNA binding of a salt tolerant dsDNase may be less sensitive to the presence of salt than a reference enzyme (e.g., a corresponding wild-type enzyme). A salt tolerant dsDNase, for example, may bind a double stranded polydeoxyribonucleic acid and/or hydrolyze one or more polydeoxyribonucleic acid substrates to yield products comprising at least one 5′-phosphorylated poly deoxyribonucleotide and/or at least one 3′-hydroxylated poly deoxyribonucleotide in the presence of at least 25 mM, at least 50 mM, at least 75 mM, at least 100 mM, at least 125 mM, or at least 150 mM salt. Catalytic activity of a salt tolerant dsDNase in the presence of 50 mM salt may be at least 40%, at least 50%, at least 60%, at least 70%, at least 80%, at least 90%, at least 95%, or at least 98% of the activity of the same salt tolerant dsDNase in the absence of salt when assayed by any method disclosed herein. A salt tolerant dsDNase in the presence of 50 mM salt may bind double stranded DNA with a dissociation constant (Kd) that is less than 10×, less than 5×, less than 2×, less that 0.5×, less that 0.25×, or less than 0.1× greater than the dissociation constant the same salt tolerant dsDNase in the absence of salt when assayed by any method disclosed herein. A salt tolerant dsDNase may display at least 20%, at least 22%, at least 24%, at least 26%, at least 28%, at least 30%, at least 35%, at least 40%, or at least 50% of its peak catalytic activity in the presence of ≤125 mM salt. In the context of salt tolerance, a salt may be a monovalent salt (e.g., NaCl), a divalent salt (e.g., MgCl₂, CaCl₂)), an organic salt (NACH₃COO), and/or an inorganic salt.

In the context of the present disclosure, “sample” refers to any material comprising or potentially comprising a target sequence including, for example, environmental materials (e.g., air, water, soil, deposits, resins, or gases, whether natural or created, and/or paper, membranes, fabrics, swabs, or other collection vehicles that have contacted such materials).

A “sample” is one or more solutions or powders that is, or may be dissolved in, an aqueous medium. A sample as described herein is used for in vitro assays, e.g., life science applications. Sample includes, but is not limited to, supernatants, isolated (fully or partially) nucleic acids, proteins, tissue supernatants, cell supernatants, cell extracts, and the like. Other sources of samples may include: blood, plasma, urine, semen, saliva, lymph fluid, meningeal fluid, amniotic fluid, glandular fluid, and cerebrospinal fluid. Samples also include fractions separated, solutions or mixtures containing known or unknown components and may be obtained at any point in time, including diagnosis, prognosis, and periodic monitoring. Specific examples of samples as described herein below for samples used in life sciences applications, e.g., removal of DNA from laboratory solutions, reverse transcription and the like.

In the context of the present disclosure, “target sequence” refers to any RNA or DNA sequence of interest.

In the context of the present disclosure, “substitution” at a position in a subject amino acid sequence refers to any difference at that position relative to the corresponding position in a reference sequence, including a deletion, an insertion, and a different amino acid, where the subject and reference sequences are otherwise at least 60% identical to each other. A substitution in a subject sequence, in addition to being different than the reference sequence, may differ from all corresponding positions in naturally occurring sequences that are at least 80% identical to the subject sequence. A substitution may be represented by a letter followed by a number, for example, E13, which indicates the position of the subject sequence corresponding to position 13 of the reference sequence and comprises an insertion, a deletion, or any amino acid other than glutamate. A substitution may be represented by a letter followed by a number followed by another letter, for example, E13K, which indicates the position 13 of the reference sequence is glutamate and the position of the subject sequence corresponding to position 13 of the reference sequence is lysine, not glutamate.

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

### Enzymes and Compositions

The present disclosure relates, in some embodiments, to dsDNases having one or more desirable properties including, for example, selective cleavage of DNA in duplex polynucleotides (e.g., DNA:DNA duplexes, DNA:RNA duplexes) relative to, for example, cleavage of dsRNA, cleavage of ssDNA, and/or cleavage of ssRNA. A composition may comprise, for example, a means for selectively cleaving a DNA strand of a DNA:RNA duplex, wherein the selective cleavage comprises cleaving ≥90% (or optionally ≥92%, ≥94%, ≥95%, ≥96%, ≥98%, or ≥99%) of the DNA strands of a DNA:RNA duplex at least once and cleaving ≤10% (or optionally ≤8%, ≤6%, ≤5%, ≤4%, ≤2%, or ≤1%) of the RNA strands even once or wherein the selective cleavage comprises cleaving ≥95% of the DNA strands at least once and cleaving ≤5% of the RNA strands even once or wherein the selective cleavage comprises cleaving ≥98% of the DNA strands at least once and cleaving ≤2% of the RNA strands even once, wherein the DNA:RNA duplex is ≤3 kb and comprises ≤10 mismatched bases (e.g., 0), 1, 2, 3, 4, 5, 6, 7, 8, 9, or 10 mismatched bases). A dsDNase additionally or alternatively may have one or more desirable properties including, for example, reduced binding affinity to actin, mucolytic activity, and/or phosphodiesterase/hydrolytic activity. Means for cleaving the DNA strand of a DNA:RNA duplex include all of the enzymes having the physical and chemical properties disclosed herein (e.g., sequences, motifs, bonds, binding properties, and other structures and features).

The present disclosure relates, in some embodiments, to an immobilized enzyme comprising a support and an enzyme immobilized thereto. For example, an immobilized dsDNase may comprise a dsDNase, a glycine-serine linker attached to the dsDNase by a peptide bond, a protein tag (e.g., a SNAP-tag) attached to the linker by a peptide bond, O⁶-benzyleguanine bound to the protein tag (e.g., SNAP-tag®); and magnetic beads having a surface modification comprising the O⁶-benzyleguanine. In some embodiments, a support of an immobilized dsDNase may comprise a magnetic bead. A magnetic bead may comprise, for example, one or more surface modifications. Surface modifications may include, for example, O⁶-benzyleguanine and/or PEG₇₅₀. In some embodiments, an immobilized enzyme may comprise a ligand (e.g., O⁶-benzyleguanine) and a receptor or tag (e.g., a SNAP-tag®) capable of binding the ligand. For example, ligands may be disposed on a support and corresponding receptors may be disposed on (e.g., covalently attached to) an enzyme to be immobilized on the support. An immobilized enzyme may comprise, in some embodiments, an enzyme (e.g., dsDNase), optionally, a first linker (e.g., a peptide linker) attached to the enzyme, a polypeptide tag (e.g., a SNAP-tag®) attached to the first linker, if present, or the enzyme, a ligand corresponding to the polypeptide tag (e.g., O⁶-benzyleguanine) attached (e.g., covalently attached) to the tag, optionally, a second linker (e.g., polyethylene glycol) attached to the ligand, and a support (e.g., a magnetic bead) attached to the second linker if present or the ligand, the structure of which may be illustrated as:

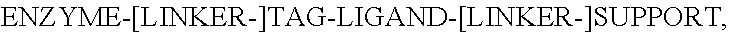

wherein dashes represent bonds (covalent or non-covalent) and brackets represent optional elements.

According to some embodiments, a dsDNase composition may comprise a dsDNase and, optionally, any of (including one or more of) a buffering agent (e.g., a storage buffer, a reaction buffer), an excipient, a salt (e.g., NaCl, MgCl₂, CaCl₂), a protein (e.g., albumin, topoisomerase, polymerase), a stabilizer, a detergent (for example, ionic, non-ionic, and/or zwitterionic detergents (e.g., octoxinol, polysorbate 20), a polynucleotide, a cell (e.g., intact, digested, or any cell-free extract), a biological fluid or secretion (e.g., mucus, pus), an aptamer, a pH indicator (e.g., azolitimin, bromocresol purple, bromothymol blue, methylene blue, cresol red, neutral red, naphtholphthalein, phenol red), a crowding agent, a sugar (e.g., a mono, di, tri, tetra, or higher saccharide), a starch, cellulose, a glass-forming agent (e.g., glycerol, raffinose, stachyose, or trehalose for lyophilization), a lipid, an oil, aqueous media, a support (e.g., a bead) and/or (non-naturally occurring) combinations thereof. Combinations may include for example, two or more of the listed components (e.g., a salt and a buffer) or a plurality of species of a single listed component (e.g., two different salts or two different sugars). According to some embodiments, dsDNase compositions may comprise (a) a dsDNase, and (b) a polynucleotide (e.g., a known dsDNase substrate, a candidate dsDNase substrate, a test sample comprising or potentially comprising a dsDNase substrate), including, for example, a double stranded DNA, a duplex polynucleotide comprising at least one DNA strand, a fluorescent probe (e.g., FIG. 2A-2D). A composition, in some embodiments, may comprise one or more modified nucleotides (e.g., base modified nucleotides, sugar modified nucleotides, and/or labeled nucleotides) and/or one or more modified nucleotide linkages (e.g., thiol linkages, azido linkages). A DNA strand and/or an RNA strand (e.g., one or both strands of a duplex dsDNase substrate) may comprise, for example, one or modified nucleotides and/or nucleotide linkages. To illustrate, a DNA:DNA duplex may comprise a first DNA strand having one or more modified nucleotides and/or one or more modified nucleotide linkages and a second DNA strand independently having one or more modified nucleotides and/or one or more modified nucleotide linkages. For example, an RNA:DNA duplex may comprise an RNA strand having one or more modified nucleotides and/or one or more modified nucleotide linkages and a DNA strand independently having one or more modified nucleotides and/or one or more modified nucleotide linkages. Modified nucleotides and/or linkages may be included, for example, to increase or decrease the stability of a duplex molecule and/or increase or decrease susceptibility of a strand to cleavage by a dsDNase. Modified nucleotides include, for example, a 2′-O—CH₃ modification, a 2′-F modification, a 2′-MOE modification, a locked nucleic acid (LNA), an unlocked nucleic acid (UNA), deoxyuridine, pseudouridine, 5-methylcytosine, 2-aminopurine, 2,6-diaminopurine, deoxyinosine, 5-hydroxybutynl-2′-deoxyuridine, 8-aza-7-deazaguanosine, 5-nitroindole.

A dsDNase composition may comprise, for example, a dsDNase variant (e.g., having an amino acid sequence of at least 85% identical to one or more of SEQ ID NO: 1-13). A dsDNase composition may be free of one or more other catalytic activities. For example, a dsDNase may be free of nucleases that cleave dsRNA, free of nucleases that cleave ssDNA, free of nucleases that cleave ssRNA (e.g., free of DNase I, RNase), free of RNA polymerase activity, free of RNA modification activity, free of protease activity, and/or free of, in each case, under desired test conditions (e.g., conditions of time, temperature, pH, salinity, model substrate and/or others), for example, conditions intended to replicate conditions of a specific use of the dsDNase composition or intended to represent conditions for a range of uses.

In some embodiments, dsDNases and compositions comprising one or more dsDNases may have any desirable form including, for example, a liquid, a gel, a film, a powder, a cake, and/or any dried or lyophilized form. A dsDNase composition may comprise a dsDNase and a support or matrix, for example, a film, gel, fabric, or bead comprising, for example, a magnetic material, agarose, polystyrene, polyacrylamide, and/or chitin. A dsDNase and compositions comprising a dsDNase may be thermostable. For example, a dsDNase or a dsDNase composition may display dsDNase activity at 25° C.-65° C.

In some embodiments, a dsDNase may be encoded by a nucleic acid sequence that, when transcribed, translated, and/or processed, results in an amino acid sequence having at least 80%, at least 85%, at least 90%, at least 93%, at least 96%, at least 97%, at least 98% or at least 99% identity to any of SEQ ID NO: 1-19. A nucleic acid encoding a dsDNase may be included in an expression cassette, expression vector, or other expressible form suitable for in vitro or in vivo expression (e.g., in E. coli or other bacteria or P. pastoris or other yeast). A nucleic acid encoding a dsDNase may be modified or optimized (e.g., codon optimized) for expression in a desired organism or cell-free expression system.

A composition having a dsDNase optionally may be free of any other enzyme or all other enzymes, according to some embodiments. For example, a composition comprising a dsDNase may lack a DNA polymerase (e.g., any specific DNA polymerase or all DNA polymerases), for example, where it may be desirable to avoid synthesizing unwanted DNA that could be digested by the dsDNase. A composition comprising a dsDNase may lack an RNA polymerase (e.g., any specific RNA polymerase or all RNA polymerases), for example, where it may be desirable to avoid synthesizing unwanted RNA that could form RNA:DNA duplexes. Similarly, a composition may lack one or more polymerase substrates (e.g., dNTPs, NTPs) to minimize or prevent undesired synthesis activity. In some embodiments, a composition comprising a dsDNase may lack a protease (e.g., any specific protease or all proteases), for example, where it is desirable to avoid inadvertent or unintended cleavage of the dsDNase and/or one or more other proteins present in the composition.

### Methods

dsDNases disclosed herein may be useful in many methods and/or workflows. dsDNases may be useful in molecular, cellular, research, sequencing, screening, diagnostic, and/or therapeutic applications. For example, dsDNases may be used in any application where it is desirable to cleave DNA in duplex form (e.g., DNA of DNA:DNA duplexes and/or the DNA strand of DNA:RNA duplexes) in a composition while leaving other components of the composition unmodified or substantially unmodified (e.g., as to structure, composition, and/or concentration). For example, dsDNases may be used in methods and/or workflows that include, for example, amplification, strand displacement, nick translation, in vitro transcription, DNA fragmentation, footprinting. PCR (e.g., RT-PCR), sequencing, and RNA purification. The present disclosure provides, in some embodiments, methods comprising contacting a dsDNase with a molecule comprising a polydeoxyribonucleotide or with a composition comprising such a molecule. For example, a method may comprise contacting a dsDNase with a composition comprising DNA in a duplex form (e.g., DNA:DNA and/or DNA:RNA duplexes). A composition contacted with a dsDNase may further comprise ssDNA, ssRNA, proteins, carbohydrates, lipids, nucleotides, buffers, salts, pH indicators, or combinations thereof.

dsDNases may be used, in some embodiments, to cleave DNA where the presence of DNA in duplex form may impair or complicate preparation or analysis of a material or sample. Methods including removal of DNA in duplex form may be regarded as “clean up” methods in both embodiments with and without additional elements and/or purposes. According to some embodiments, a method of preparing a composition comprising DNA for analysis may comprise contacting the composition with a dsDNase under conditions that permit (e.g., favor) cleavage a DNA strand in duplex form, if present.

The present disclosure relates, in some embodiments, to methods comprising contacting a dsDNase with a composition comprising DNA in duplex form and comprising RNA (e.g., ssRNA, DNA:RNA duplexes, and/or RNA:RNA duplexes) to digest the duplex DNA molecules present without substantially modifying or without modifying the RNA. For example, ssRNA and RNA:RNA duplexes, if present, may retain their size, conformation, sequence, among other properties while the RNA strand of a DNA:RNA duplex, if present, may retain its size, sequence and other properties, but change conformation from duplex to single-stranded form as its paired DNA strand is digested away.

Such contacting may be included in methods for cleaning up RNA after isolation from a cell or tissue, after in vitro transcription, prior to elution from a solid support, and/or prior to RT-PCR. The RNA may remain unaltered or substantially unaltered. For example, contacting a dsDNase with a composition comprising RNA and duplex DNA may result in a reaction mixture/product composition comprising over 50%, over 80%, over 85%, over 90%, over 95%, or over 99% (optionally, in each case, weight percent or molar percent) of the starting, intact RNA (i.e., the starting polyribonucleic acid) and/or less than 50%, less than 25%, less than 10%, less than 5%, less than 1%, less than 0.5%, or less than 0.1% (optionally, in each case, weight percent or molar percent) of the starting DNA (i.e., the starting polydeoxyribonucleic acid). As one of ordinary skill in the art having the benefit of this disclosure will appreciate, monomeric nucleotide units in a polydeoxyribonucleic acid or a polyribonucleic acid are expected to retain their identity as DNA or RNA without regard to whether the polymer of which they are a part is or is not a substrate for a dsDNase. In this context, assessing the percentage of an RNA or DNA in a reaction mixture/product composition relates to the degree to which the initial polymeric nucleic acid is present, not the extent to which monomeric building blocks of such initial polymer remain. For example, a 100 nt polyU RNA strand in an RNA:DNA duplex with a 100 nt polyA strand contacted with a dsDNase may produce a 100 nt ssRNA and 100 adenine monomers. The RNA strand in this context remains “unaltered”, “intact” whereas the DNA strand cannot be said to remain “unaltered” or “intact” even though the adenine monomers retain their identity as DNA molecules.

In some embodiments, catalytic activity of a dsDNase may be tunable. For example, a method may include contacting a dsDNase with a nucleic acid composition under conditions selected to increase (e.g., maximize) hydrolytic activity, increase (e.g., maximize) selectivity, and/or reduce (e.g., minimize) cleavage of duplex DNA comprising mismatches. For example, a method may include selecting the dsDNase, the reaction temperature, the salt ions, the salt concentration, and/or buffering agent to produce the desired reaction products.

A method may include fracturing one or more cells to form the composition comprising DNA and RNA, wherein at least a portion of the DNA comprises cellular DNA and at least a portion of the RNA comprises cellular RNA. The size of digestion products and/or degree of digestion may be managed, for example, by selecting the dsDNase with a desired activity, increasing or decreasing the concentration of the selected dsDNase, increasing or decreasing the incubation time or temperature, increasing or decreasing calcium concentration, increasing or decreasing magnesium concentration and/or increasing or decreasing salt concentration. In some embodiments, methods may be adapted to digest DNA as fully as practicable or digest DNA (non-specifically) into fragments within a selected range of sizes.

The present disclosure relates, in some embodiments, to methods comprising contacting a dsDNase with a composition comprising DNA in duplex form and a protein (or another non-DNA molecule of interest) to digest the DNA molecules present without modifying the protein (or another non-DNA molecule of interest). Such contacting may be included in methods for cleaning up protein after isolation from a cell or tissue, preparing protein samples for separation on 2-D gels, and/or identifying protein binding sequences on DNA (DNase I footprinting). The protein may remain unaltered or substantially unaltered. For example, contacting a dsDNase with a composition comprising DNA and a protein (or another non-DNA molecule of interest) may result in a product composition comprising over 50%, over 80%, over 85%, over 90%, over 95%, or over 99% of the starting, intact protein (or another non-DNA molecule of interest) and/or less than 50%, less than 25%, less than 10%, less than 5%, less than 1%, less than 0.5%, or less than 0.1% of the starting, DNA in duplex form. A method may include fracturing one or more cells to form the composition comprising DNA in duplex form and, optionally, a ssDNA, a protein, or a non-DNA molecule of interest), wherein at least a portion of the DNA in duplex form comprises cellular DNA. The optional ssDNA, protein, or non-DNA molecule may be a cellular ssDNA, a cellular protein, or a cellular non-DNA molecule.

In some embodiments, a dsDNase may be contacted with a composition comprising DNA in duplex form (e.g., genomic fragments or other long (≥10 kb) DNA fragments) to digest such DNA. Such contacting may be included in methods for creating a fragmented DNA library and methods of cell culture preparation (e.g., tissue disaggregation), cultivation, manipulation, and storage to reduce or prevent cell clumping.

A dsDNase may be used in connection with in vitro transcription (IVT), according to some embodiments. IVT methods may include contacting a DNA template (e.g., a double stranded DNA comprising a coding sequence and an expression control sequence operably linked to the coding sequence) with an RNA polymerase (e.g., T7 RNA polymerase) optionally in the presence of NTPs, salt, and/or a reaction buffer to form a transcription product composition comprising a transcription product (e.g., RNA) and the DNA template. IVT methods, in some embodiments, may comprise contacting a dsDNase with DNA in duplex form (e.g., duplex DNA:DNA comprising the DNA template with its complementary DNA strand or duplex DNA:RNA comprising the template and the nascent transcript) to digest the DNA and form a digested composition comprising DNA digestion products and intact transcription product (e.g., single-stranded transcript and/or subsequently translated protein). IVT methods may comprise separating the transcription product from one or more of the other components of the transcription product composition or the digested composition, for example, the DNA template, DNA template digestion products, the RNA polymerase, NTPs, salt, and reaction buffer. Techniques for such separation include column purification, phase separation (e.g., phenol-chloroform), and fractionation (e.g., size, charge, hydrophobicity, polarity, and ratios thereof) among others.

A method may include transcribing a DNA template to form the composition comprising DNA and RNA, wherein at least a portion of the DNA comprises the DNA template and at least a portion of the RNA comprises RNA arising from the transcription. A method may comprise contacting a dsDNase with a composition comprising DNA in duplex form and RNA to produce a product solution comprising RNA and optionally less than 5%, less than 4%, less than 3%, less than 2%, less than 1%, less than 0.5% of the intact DNA in the starting composition.

The amount of RNA (e.g., transcription product) remaining after contact with the dsDNase may be assessed by available methods including, for example, RT-qPCR. The amount of DNA template remaining after contact with the dsDNase may be assessed by available methods including, for example, qPCR. In some embodiments, hydrolysis of DNA may be detected by including in a reaction mixture or contacting a reaction mixture with a pH indicator.

According to some embodiments, contacting a polydeoxyribonucleic acid (e.g., the template DNA for IVT) with a dsDNase may (further) comprise contacting the polydeoxyribonucleic acid with the dsDNase in the presence of at least 10 mM salt, at least 25 mM salt, at least 50 mM salt, at least 100 mM salt, at least 150 mM salt, at least 200 mM salt, at least 250 mM salt, at least 300 mM salt, and/or at least 350 mM salt. In each case, salt present may be a single salt species or a mixture of salts. In each case, salt present may comprise monovalent and/or divalent salts.

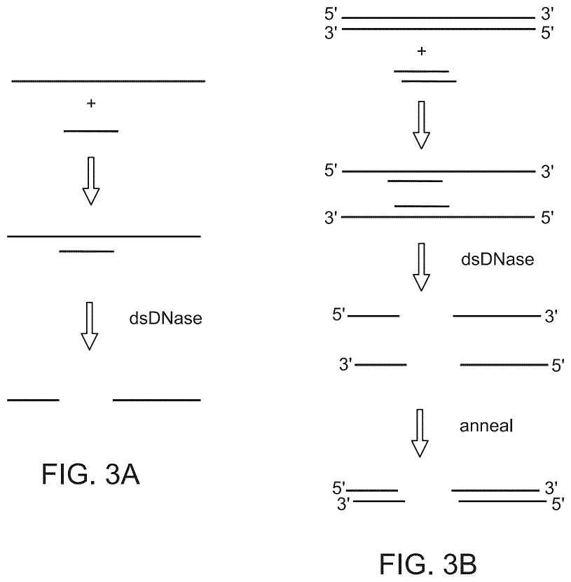

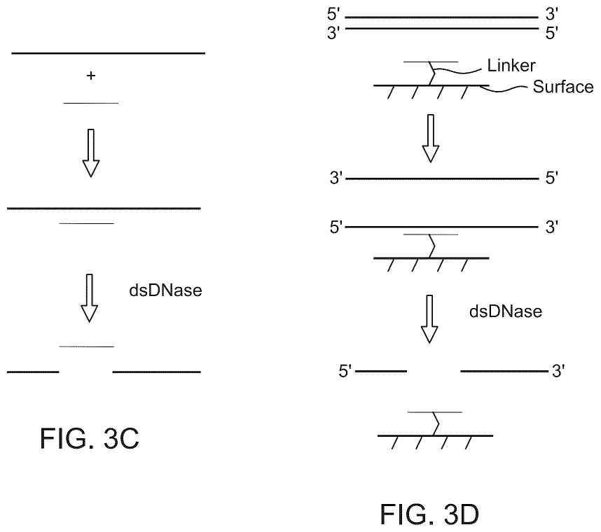

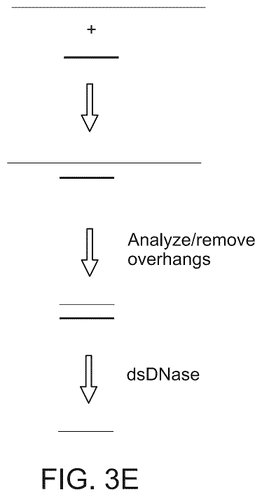

In some embodiments, a dsDNase may be used for (optionally) programmable cleavage of a DNA. For example, a method may comprise contacting a probe DNA comprising a sequence complementary to a target sequence with one or more DNA molecules (e.g., a DNA library or other population of DNAs) comprising (or potentially comprising) a DNA having the target sequence to form a homoduplex comprising the probe DNA and the DNA comprising the target sequence. In some embodiments, a probe DNA sequence complementary to a target may be a discrete, known sequence (e.g., for programmable cleavage), a degenerate sequence, or a random sequence (e.g., a population of probe DNA molecules of sufficient numerosity to contain about or at least one copy of each possible sequence). In some embodiments, a probe DNA may be linked to a label (e.g., an affinity tag or a fluorophore) or surface (e.g., a support, a bead, a matrix, a column), optionally, by a linker, a 5′ end, a 3′ end, or combinations thereof. A homoduplex may comprise further a 5′ DNA overhang, a 3′ DNA overhang, or combinations thereof. FIG. 3A illustrates an example method comprising contacting a first (optionally longer) DNA strand and a second (optionally shorter) DNA strand to form a homoduplex comprising a duplex portion and one or more (e.g., two) overhangs. In some embodiments, the first strand or the second strand may comprise a DNA sequence complementary to a target sequence. For example, a second strand may be contacted with the first strand (or a library of first strands (each having the same or different sequences) to form a duplex with the target sequence (if present) in a first strand. A method may further comprise contacting the homoduplex with a dsDNase to form cleavage products. As illustrated, the dsDNase may cleave (digest) away both DNA strands of the duplex portion. In some embodiments, a first strand or a second strand may be linked to a surface (e.g., a bead, a plate, or a well), optionally through a linker.

FIG. 3B illustrates an example method comprising contacting a first (optionally longer) dsDNA comprising a top and bottom strand and a second (optionally shorter) dsDNA comprising a top and bottom strand to form a homoduplex comprising the top strand of the first DNA and bottom strand of the second DNA and a homoduplex comprising the bottom strand of the first DNA and the top strand of the second DNA. Each homoduplex may comprise a duplex portion and one or more (e.g., two) overhangs. A method may further comprise contacting one or both homoduplexes with a dsDNase to form cleavage products. Cleavage products may be treated (e.g., contacted with a dsDNase inhibitor, heated, and/or washed) to inactivate and/or remove the dsDNase and form treated cleavage products. Treated cleavage products may be annealed to form duplex DNA products.

In some embodiments, a method may comprise contacting a probe RNA comprising a sequence complementary to a target sequence with one or more DNA molecules (e.g., a DNA library or other population of DNAs) comprising (or potentially comprising) a DNA having the target sequence to form a heteroduplex comprising the probe RNA and the DNA comprising the target sequence. In some embodiments, a probe RNA sequence complementary to a target may be a discrete, known sequence (e.g., for programmable cleavage), a degenerate sequence, or a random sequence (e.g., a population of probe RNA molecules of sufficient numerosity to contain about or at least one copy of each possible sequence). In some embodiments, a probe RNA may be linked to a label (e.g., an affinity tag or a fluorophore) or surface (e.g., a support, a bead, a matrix, a column), optionally, by a linker, a 5′ end, a 3′ end, or combinations thereof. A heteroduplex may comprise further a 5′ DNA overhang, a 3′ DNA overhang, or combinations thereof. FIG. 3C illustrates an example method comprising contacting a first (optionally longer) DNA strand (thick bar) and a second (optionally shorter) RNA strand (thin bar) to form a heteroduplex comprising a duplex portion and one or more (e.g., two) overhangs. In some embodiments, the second strand may comprise an RNA sequence complementary to a target sequence. For example, a second strand may be contacted with the first strand (or a library of first strands (each having the same or different sequences) to form a duplex with the target sequence (if present) in a first strand. A method may further comprise contacting the heteroduplex with a dsDNase to form cleavage products. As illustrated, the dsDNase may cleave (digest) away the DNA strand of the duplex portion. In some embodiments, a first strand or a second strand may be linked to a surface (e.g., a bead, a plate, or a well), optionally through a linker.

FIG. 3D illustrates an example method comprising contacting a dsDNA comprising a top strand and a bottom strand and a ssRNA (optionally shorter than the dsDNA) to form a heteroduplex comprising the top strand of the dsDNA and the ssRNA. A heteroduplex may comprise a duplex portion and one or more (e.g., two) overhangs. As illustrated, the ssRNA is linked to a surface by an optional linker. The bottom strand may be separated from the heteroduplex, for example, by washing the surface. A method may include contacting the heteroduplex with a dsDNase to form cleavage products. Cleavage products may include one or more fragments of the top strand (e.g., a 5′ fragment and a 3′ fragment).

In some embodiments, a method may comprise masking and unmasking a portion of an RNA molecule. For example, a method may comprise contacting an RNA strand with a DNA strand to form a heteroduplex comprising a duplex (masked) portion and optionally further comprising a 5′ RNA overhang, a 3′ RNA overhang, a 5′ DNA overhang, a 3′ DNA overhang, or combinations thereof. A method optionally may comprise separating heteroduplexes from other polynucleotides present (e.g., single stranded DNA, single stranded RNA, double stranded DNA, and/or double stranded RNA), analyzing and/or removing overhangs, if any, and/or combinations thereof. A method may comprise contacting at least the duplex portion with a dsDNase to form an unmasked RNA. For example, a DNA strand may be immobilized on a surface (e.g., a support, a bead, a matrix, a column). An RNA strand (e.g., an RNA strand included in a population of polynucleotides) comprising a sequence complementary to the sequence of the immobilized DNA strand may be contacted with the immobilized DNA strand to form a heteroduplex. Following any desired processing, analysis, or purification, at least the duplex portion of the heteroduplex may be contacted with a dsDNase to release (unmask) the RNA portion of the duplex.

FIG. 3E illustrates an example method comprising contacting a DNA strand (thick bar) with an RNA strand (thin bar) to form a heteroduplex comprising a duplex portion and one or more (e.g., two) overhangs. A method may further comprise cleaving overhangs (pictured) and/or purifying and/or analyzing the heteroduplex or any portion thereof. A method may further comprise melting the heteroduplex or any portion thereof to form separate strands and optionally analyzing (e.g., sequencing) or purifying one or more of the separated strands.

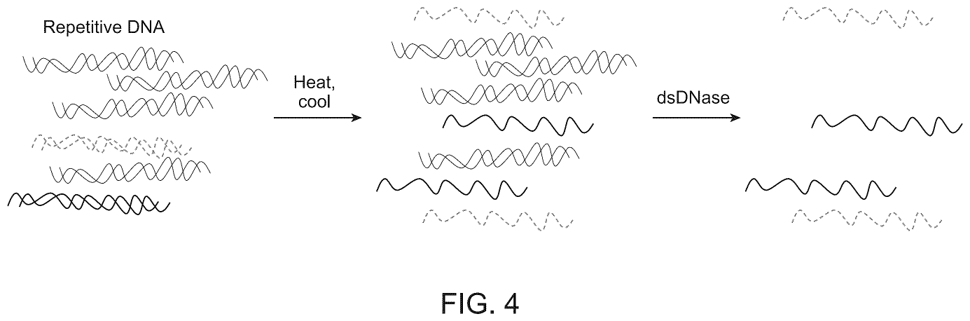

In some embodiments, dsDNases may be used to reduce the number of repetitive DNA sequences in a population of DNA molecules (e.g., genomic DNA. cDNA). In this context, repetitive sequences may include two or more sequences (e.g., ≤10 nucleotides long, 10-25 nucleotides long, 10-200 nucleotides long, ≤50 nucleotides long, 50-2000 nucleotides long) present in a population of polynucleotides that are identical or substantially identical (e.g., over 97%, over 98 or over 99% sequence identity). For example, a method may include denaturing (e.g., heating) and reannealing (e.g., cooling) a population of DNA molecules comprising repetitive sequences to form a population of partially annealed DNA as shown in FIG. 4. Conditions may be chosen (e.g., heating to ≥90° C. for 5-600 seconds and holding at 65-70° C. for 1-6 hours) to favor reannealing of repetitive sequences (compared to annealing of non-repetitive sequences). A method may further include contacting the population of partially annealed DNA with a dsDNase to form a duplex DNA cleavage composition comprising duplex DNA cleavage products (e.g., mononucleotides and optionally oligonucleotides ≤15 nts) and a population of DNA molecules having fewer repetitive sequences than the starting population. In some embodiments, denaturing, annealing and contacting steps may be repeated as desired, for example, where the starting population comprises highly redundant sequences. Without being limited to any mechanism of action, repetitive sequences may anneal more quickly and/or with higher frequency than non-redundant sequences upon cooling since any one redundant sequence has more (e.g., many more) sequences with which it may hybridize (even if not perfectly complementary).

FIG. 4 shows an example of a method of using a dsDNase to reduce or remove repetitive DNA in a population of DNA molecules. A population of DNA molecules may be heated to separate the strands of duplex DNA and then cooled. DNA strands comprising repetitive sequences (thin) may reanneal more readily (e.g., with higher frequency) than DNA strands comprising more unique sequences (dashed and thick lines), which may tend to remain in single-stranded form. This resulting mixture of duplex and single-stranded DNA may be contacted with a dsDNase to digest the duplex (repetitive) DNA present while leaving intact the single-stranded (unique) DNA present.

The present disclosure further provides methods for using a dsDNase to detect the presence of a target (e.g., RNA and/or DNA) sequence in a population of molecules (e.g., RNA and/or DNA). A detection method may be performed, according to some embodiments, in a single “pot” including in a single reaction vessel, tube, well, or other container, or on a single surface, in a single column. Methods may comprise conditionally forming a duplex comprising at least one DNA strand when a target is present and contacting the duplex with a dsDNase to form dsDNase cleavage products. In some embodiments, the duplex may comprise the target sequence, a reporter, or both the target sequence and a reporter. For example, a method may comprise contacting (i) a sample (e.g., a biological material or fluid, an environmental material or fluid, a polynucleotide library) comprising or potentially comprising a target polynucleotide sequence; (ii) a dsDNase; and (iii) a reporter comprising a single-stranded DNA having two ends (e.g., a 5′ end and a 3′ end), wherein one end comprises a fluorophore, the other comprises a quencher corresponding to the fluorophore, and the single-stranded DNA is complementary to the target sequence, to form a reaction mixture. Without being limited to any specific mechanism of action, contacting may be performed to permit hybridization of the reporter to the target sequence (if present) to form a duplex that is susceptible to cleavage by the dsDNase. If the target is DNA, the dsDNase would cleave both the reporter strand of the duplex and the target sequence strand of the duplex. If the target is RNA, the dsDNase would cleave the reporter strand. Cleavage of the reporter strand allows the fluorophore to separate from the quencher and, under appropriate illumination, fluoresce.

The present disclosure relates to methods comprising contacting an immobilized enzyme to a substrate to form a product, separating the immobilized enzymes from the product, and optionally contacting the immobilized enzyme to more substrate to form more product. For example, a method may comprise contacting a first portion of a double stranded DNA substrate with an immobilized enzyme (e.g., dsDNase) to produce double stranded DNA cleavage products, separating the immobilized enzyme from the double stranded DNA cleavage products to form separated immobilized enzyme and separated double stranded DNA cleavage products, and/or contacting a second portion of the double stranded DNA substrate with the separated immobilized enzyme to produce more double stranded DNA cleavage products. In some embodiments, a method may further comprise repeating the contacting and separating steps (e.g., 2-50 cycles). A method may further comprise combining the separated double stranded DNA cleavage products with the more double stranded DNA cleavage products to produce pooled products.

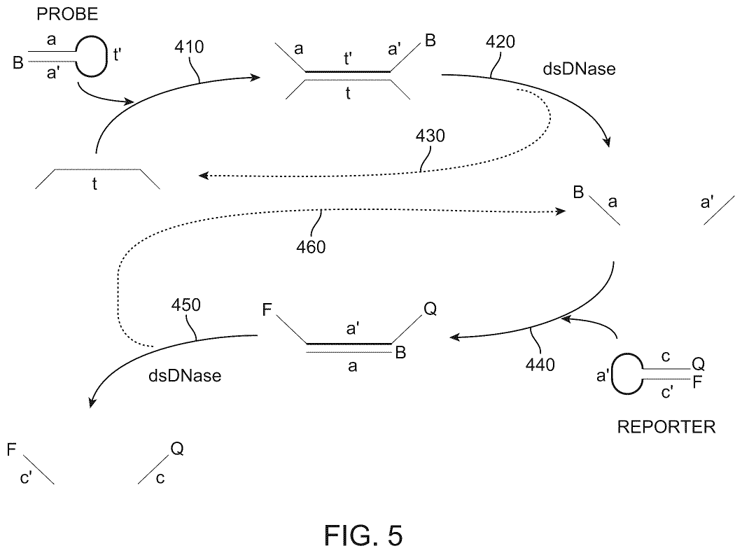

In some embodiments, a dsDNase may be used to screen for the presence of a polynucleotide of interest. For example, a dsDNase may be used to detect the presence of a target sequence in a population of RNA molecules (FIG. 5). An RNA molecule comprising a target sequence (t) (e.g., an RNA molecule in a library or population of RNA molecules) is contacted 410 with a hairpin probe. A hairpin probe may include a DNA loop comprising a sequence (t′) complementary to the target and an RNA stem comprising hybridized strands, each strand comprising a complementary sequence (a or a). Contacting 410 the RNA molecule and the probe may form a heteroduplex comprising sequences t (RNA) and t′ (DNA). The heteroduplex may be contacted 420 with a dsDNase to produce cleavage products comprising the RNA molecule comprising the target sequence (t) and fragments of the probe comprising sequences a (fragment a) and a′ (fragment a). In some embodiments, a method may include signal amplification 430 wherein the released RNA molecule comprising sequence t contacts another hairpin probe molecule. Fragment a of the probe may contact 440 a hairpin reporter. A hairpin reporter may include a DNA loop comprising the a′ sequence and an RNA stem comprising strands, each comprising a sequence complementary to the other (c and c′), with one strand further comprising a fluorophore (F) and the other strand further comprising a quencher (Q) to form a heteroduplex, the heteroduplex comprising probe fragment a (RNA) and reporter loop a′ (DNA). The duplex may be contacted 450 with a dsDNase to produce cleavage products comprising fragment a of the probe and fragments of the reporter comprising sequences c (fragment c) and c′ (fragment c′). A method may include signal amplification 460 wherein the released probe fragment a contacts another hairpin reporter molecule. A fluorescent signal may result from separation of the fluorophore and the quencher (under appropriate illumination). Fluorescence may be measured without further processing and the presence or absence of a target sequence in the population of RNA molecules may be recognized by absence, presence, and or intensity of any fluorescent signal.

In some embodiments, a method may include one or more measures to reduce background fluorescence, if present. For example, if the hairpin probe stem arm comprising sequence a contacts the reporter loop comprising sequence a′, the two may form a heteroduplex susceptible to dsDNase cleavage, which could result in fluorescence not attributable to the presence of a target sequence. A hairpin probe may include a DNA loop comprising a sequence (t′) complementary to the target and an RNA stem comprising hybridized strands, each strand comprising a complementary sequence (a or a), wherein the strand comprising the a sequence is linked to a separation tag (e.g., an affinity tag, a support, a bead, a matrix, a column). A method may include, in some embodiments, removing or separating the separation tag. For example, a method may be performed with a hairpin probe linked to a separation tag comprising a magnetic bead and separating the separation tag may include magnetically gathering the beads to form a pellet and a supernatant and optionally separating the pellet and supernatant. The pellet may comprise fragment a′ and remaining unreacted probe, if any. Removal of unreacted probe may limit direct interaction between the probe and reporter, reducing fluorescence not attributable to the presence of the target.

In some embodiments, a probe and a reporter may be spatially separated, for example, immobilizing each on spatially separate locations (e.g., on a surface). For example, a method may comprise contacting an RNA molecule comprising a target sequence (t) with an immobilized hairpin probe, the hairpin probe including a DNA loop comprising a sequence (t′) complementary to the target and an RNA stem comprising hybridized strands, each strand comprising a complementary sequence (a or a′). Contacting the RNA molecule and the probe may form a heteroduplex comprising sequences t (RNA) and t′ (DNA), which may be contacted with a dsDNase to produce cleavage products comprising the RNA molecule comprising the target sequence (t) and fragments of the probe comprising sequences a (fragment a) and a′ (fragment a′). In some embodiments, a method may include signal amplification wherein the released RNA molecule contacts another hairpin probe molecule. Fragment a of the probe may contact an immobilized hairpin reporter that includes a DNA loop comprising the a sequence and an RNA stem comprising strands, each strand comprising a sequence complementary to the other (c and c′), with one strand further comprising a fluorophore (F) and the other strand optionally further comprising a quencher (Q). Contacting fragment a and the reporter may form a heteroduplex, the heteroduplex comprising probe fragment a (RNA) and reporter loop a′ (DNA). The duplex may be contacted with a dsDNase to produce cleavage products comprising fragment a of the probe and fragments of the reporter comprising sequences c (fragment c) and c′ (fragment c′). A method may include signal amplification wherein the released probe fragment a contacts another hairpin reporter molecule. A fluorescent signal may result from separation of the fluorophore and the optional quencher (under appropriate illumination). Fluorescence may be measured without further processing and the presence or absence of a target sequence in the population of RNA molecules may be recognized by absence, presence, and or intensity of any fluorescent signal.

In some embodiments, a reporter, which may or may not comprise a quencher, may be immobilized on a surface. It may be desirable to move a reaction mixture away from an immobilized reporter. A method may include, for example, contacting an RNA molecule and a dsDNase with a surface comprising an immobilized probe and an immobilized reporter (e.g., in spatially separate locations) to form a reaction mixture. For example, a method may include drawing a fluid comprising an RNA molecule and a dsDNase (optionally, with one or more additional components including buffer, salt, and detergent among others) into a pipet tip, the inner surface of which comprises an immobilized probe and an immobilized reporter, the probe and the reporter in spatially separate portions of the inner surface to form a reaction mix. A method may further include, expelling the reaction mix, for example, into a cuvette or well, at a desired time and measuring the fluorescence of the expelled reaction mix.

The present disclosure further relates to methods of making a dsDNase. For example, a dsDNase may be produced by in vitro transcription and/or in vitro translation (e.g., PURExpress®, New England Biolabs, Inc.). In some embodiments, a dsDNase may be produced in vivo. For example, a method may include (a) culturing a host cell (e.g., a bacterial host cell, a yeast host cell, a mammalian host cell, a plant host cell) comprising an expression vector or expression cassette comprising a nucleic acid sequence having at least 80%, at least 85%, at least 90%, at least 93%, at least 96%, at least 97%, at least 98% or at least 99% identity to a sequence (e.g., a codon optimized sequence) encoding any of the amino acid sequences SEQ ID NO: 1-19 operably linked to an expression control sequence to produce a cultured host cell composition comprising the dsDNase, and (optionally) isolating the dsDNase from the cultured host cell composition (e.g., culture supernatant, culture lysate, culture cell paste).

One of ordinary skill in the art having the benefit of Applicant's disclosure would recognize that a dsDNase may be used in many other methods and workflows. For example, a dsDNase may be used to deplete one or more polynucleotides present in a population of polynucleotides. Depletion methods in which a dsDNase disclosed herein may be used include any of the methods of Archer et al., “Selective and flexible depletion of problematic sequences from RNA-seq libraries at the cDNA stage”, BMC Genomics 15:401, Bogdanova et al., “DSN depletion is a simple method to remove selected transcripts from cDNA populations”, Mol. Biotechnol. 41(3):247-253, Wu et al., “Recent advances in duplex-specific nuclease-based signal amplification strategies for microRNA detection”, Biosensors and Bioelectronics 165: 112449, Xiao et al., “High-throughput RNA sequencing of a formalin-fixed, paraffin-embedded autopsy lung tissue sample from the 1918 influenza pandemic”, Journal of Pathology 229(4):535-545, Yi et al., “Duplex-specific nuclease efficiently removes rRNA for prokaryotic RNA-seq”, Nucleic Acids Research 39(20):e140, and Zhulidov et al., “Simple cDNA normalization using kamchatka crab duplex-specific nuclease”, Nucleic Acids Research 32(3):e37.

### Kits

The present disclosure further relates to kits including dsDNases. For example, a kit may include a dsDNase and one or more of dNTPs, rNTPs, primers, other enzymes (e.g., polymerases, single-stranded binding proteins, helicases, argonauts), buffering agents, pH indicators, or combinations thereof. A dsDNase may be included in a storage buffer (e.g., comprising a buffering agent and comprising or lacking glycerol). A kit may include a reaction buffer which may be in concentrated form, and the buffer may contain additives (e.g., glycerol), salt (e.g., KCl), reducing agent, EDTA or detergents, among others. A kit comprising dNTPs may include one, two, three of all four of dATP, dTTP, dGTP and dCTP. A kit comprising rNTPs may include one, two, three of all four of rATP, rUTP, rGTP and rCTP. A kit may further comprise one or more modified nucleotides. A kit may optionally comprise one or more primers (random primers, bump primers, exonuclease-resistant primers, chemically modified primers, custom sequence primers, or combinations thereof). One or more components of a kit may be included in one container for a single step reaction, or one or more components may be contained in one container, but separated from other components for sequential use or parallel use. The contents of a kit may be formulated for use in a desired method or process.

A kit is provided that contains: (i) a dsDNase; and (ii) a buffer. A dsDNase may be present in a dried or lyophilized form or may be included in a buffer (e.g., a storage buffer or a reaction buffer in concentrated form). A kit may contain a dsDNase in a tube or mastermix suitable for receiving and transcribing a template nucleic acid. For example, a dsDNase may be deposited in the cap of a tube while components for transcribing a template nucleic acid are deposited in the body of the tube. As desired, for example, upon completion of the transcription reaction, the tube may be tapped, shaken, turned, spun, or otherwise moved to contact the deposited dsDNase with the transcription reaction mixture. A reaction buffer may include non-ionic, ionic (e.g., anionic or zwitterionic) surfactants and/or crowding agents. A kit may include a dsDNase and the reaction buffer in a single tube or in different tubes and, if included in a single tube, the dsDNase and the buffer may be present in the same or separate locations in the tube.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or sub-packaging) etc. Instructions may be present as an electronic storage data file residing on a suitable computer readable storage medium (e.g., a CD-ROM, a flash drive). Instructions may be provided remotely using, for example, cloud or internet resources with a link or other access instructions provided in or with a kit.

### EXAMPLES

Some specific example embodiments may be illustrated by one or more of the examples provided herein. As example embodiments, generalization of all conditions and results is regarded to be possible (unless stated otherwise) and contemplated to be within the scope of the disclosure.

### Example 1: Cloning DNase Genes in Pichia pastoriis

Candidate dsDNase genes were cloned into either pD912(AOX) or pD912(GAP) with an N-terminal α-mating factor secretion signal (SEQ ID NO:20) and C-terminal linker and His6 tag (amino acid sequence NSAVDHHHHHH (SEQ ID NO: 21). The pD912(GAP) plasmid is a derivative of pD912(AOX), in which the AOX1 promoter is replaced with a 484 bp fragment containing the constitutive GAP promoter from Pichia pastoris. dsDNase plasmids were linearized by amplifying with primers pD912casF (GCTCATTCCAATTCCTTCTATTAG (SEQ ID NO: 22)) and pD912casR (GAGCTCCAATCAAGCCCAATAAC (SEQ ID NO: 23). The resulting cassette was used to transform electrocompetent P. pastoris MutS cells. Transformants were selected on yeast peptone dextrose (YPD) agar medium supplemented with 1 M sorbitol and 500 μg/mL Zeocin (Teknova).

Several colonies of each clone were grown for 48 h at 30° C. in BMGY-Buffered Glycerol Complex Medium (Teknova) (1% yeast extract, 2% tryptone, 1.34% yeast nitrogen base (YNB) without amino acids with ammonium sulfate, 0.0004% biotin, 1% glycerol, 100 mM potassium phosphate, pH 6.0). Cultures were centrifuged and the supernatants were analyzed by SDS-PAGE and screened for dsDNase activity as described below. Active clones were confirmed by Sanger sequencing of the inserted dsDNase gene. Insert copy number and integration site were later determined by Nanopore whole genome sequencing. Genomic DNA libraries were prepared using the Rapid Barcoding Sequencing Kit (ONT) and pooled for sequencing on a GridION sequencer (ONT).

For expression studies using pD912(AOX), individual colonies were grown for 48 h in BMGY medium. After this initial growth phase, cultures were centrifuged and resuspended in ⅕ the original culture volume in BMMY-Buffered Methanol Complex Medium (Teknova) (1% yeast extract, 2% tryptone, 1.34% yeast nitrogen base (YNB) without amino acids with ammonium sulfate, 0.0004% biotin, 0.5% methanol, 100 mM potassium phosphate, pH 6.0). These cultures were grown overnight at 30° C. before harvesting supernatants by centrifugation. dsDNase expression and activity was assessed as described above.

### Example 2: DNase Purification

Candidate dsDNases were expressed with a C-terminal His6 tag to facilitate purification. Cultures expressing the gene of interest were grown as described above and supernatants were harvested by centrifugation. The fresh supernatants were either concentrated and resuspended in IMAC binding buffer [50 mM Tris pH 8, 300 mM NaCl], or diluted directly in IMAC binding buffer, before loading on NEBExpress Ni resin. Candidate dsDNases were eluted from the resin using the same buffer containing 300 mM imidazole. Fractions containing the dsDNase were pooled, concentrated, and dialyzed into storage buffer (10 mM Tris pH 7.5, 500 mM NaCl, 1 mM DTT, 0.1 mM EDTA, 50% glycerol).

### Example 3: DNase Activity Assays

ssDNA and dsDNA FAM-labeled probes (FIGS. 1A, 1B) were synthesized by Integrated DNA Technologies (Coralville, IA). Both probes contained a 5′-FAM label and a 3′ Black Hole Quencher-1. Probes were individually resuspended at 100 μM in IDT duplex buffer (30 mM HEPES pH 7.5, 100 mM potassium acetate). The dsDNA probe was annealed by heating in a thermal cycler to 95° C. for 3 min. and then cooling to room temperature over 30 min.

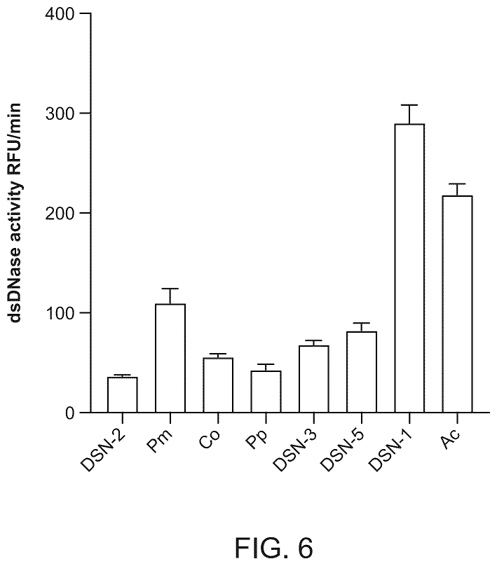

P. pastoris culture supernatants of Example 1 and purified proteins prepared according to Example 2 were assayed for DNase activity. Culture supernatants were diluted 100-fold in assay buffer (25 mM Tris pH 7.5, 5 mM MgCl₂) immediately before the assay. 5 μl of diluted culture supernatant was assayed in the same buffer in a final reaction volume of 30 μl containing 3.33 μM ssDNA or dsDNA probe in a black half-area 96-well plate (Costar 3694). Reactions were mixed by pipetting up and down and immediately incubated at 30° C. in a Biotek Synergy Neo2 plate reader. Fluorescence was measured every 18 seconds over five minutes, using an excitation wavelength of 485 nm and an emission wavelength of 528 nm. Fluorescence increased linearly over this time frame, and enzyme activities (RFU/min) were determined by calculating the slope over the first two minutes of the reaction. Results are shown in FIG. 6 for example culture supernatants of clones expressing dsDNases of P. monodon (Pm), C. opilio (Co), P. pelagicus (Pp), and A. chinensis (Ac) and non-naturally occurring sequences DSN-2, DSN-3, DSN-5, and DSN-1.

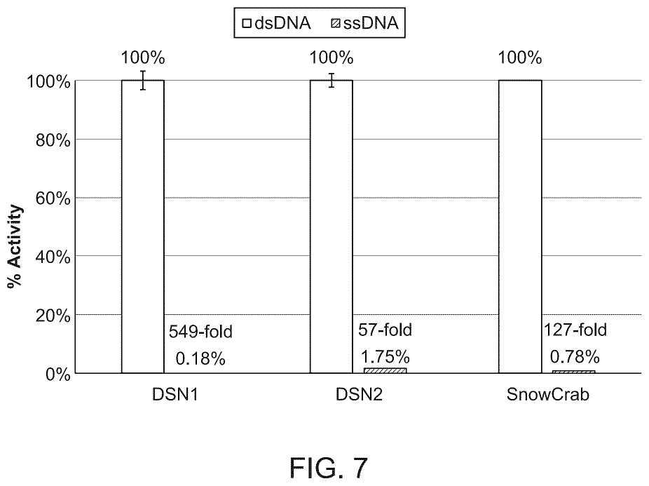

Purified proteins were combined with prepared ssDNA and dsDNA probes (5 μM) and buffer (10 mM Tris-HCl, pH 7.9, 10 mM MgCl₂, 50 mM NaCl, 100 μg/ml recombinant albumin) and incubated at 30° ° C. in a black half-area 96-well plate (Costar 3694). Fluorescence was measured every 10 seconds for a total of 2 minutes. Activity on a dsDNA substrate (35 nt dsDNA hairpin, white bars) or ssDNA substrate (15 nt ssDNA, black bars) was measured by monitoring the increase in fluorescence as the substrate is cleaved. Fluorescence increased linearly over this time frame, and enzyme activities (RFU/min) were determined by calculating the slope between 10-70 seconds of the reaction. DSN-2, DSN-1, and Snow Crab enzymes all displayed increased specificity for dsDNA over ssDNA (57 to 549-fold increased specificity for dsDNA compared to ssDNA) (FIG. 7).

### Example 3B: DNase Activity—Substrate Length

Purified proteins were combined with fluorescently labeled dsDNA molecules (2 pmol) of various sizes (either 60, 21, 20 18, 15, 12, 10, 9 or 5 bps) and buffer (10 mM Tris-HCl, pH 7.9, 10 mM MgCl₂, 50 mM NaCl, 100 μg/ml recombinant albumin) and incubated at 37° C. in a 96-well plate for 15 minutes. Samples were quenched on ice before cleaved fragments were analyzed by capillary electrophoresis on an ABI 3730 sequencer. Only dsDNAs larger than 9 base pairs in length were cleaved by DSN1 and DSN2.

### Example 4: DNase Activity in the Presence of Divalent Cations

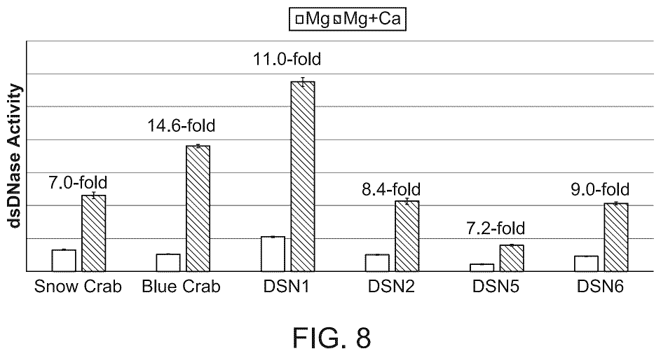

DNase activity was measured by monitoring the increase in fluorescence when a dsDNA hairpin substrate (5 μM) prepared according to Example 3 is cleaved by a purified dsDNase in 1× Mg²⁺ Reaction Buffer (10 mM Tris-HCl, pH 7.9, 50 mM NaCl, 10 mM MgCl₂, 100 μg/ml recombinant albumin) or 1× Mg²⁺/Ca²⁺ Reaction Buffer (10 mM Tris-HCl, pH 7.6, 2.5 mM MgCl₂, 0.5 mM CaCl₂, 100 μg/ml recombinant albumin) at 30° C. in a black half-area 96-well plate (Costar 3694). Fluorescence was measured every 10 seconds for a total of 2 minutes. Activity was measured by monitoring the increase in fluorescence as the substrate is cleaved. Fluorescence increased linearly over this time frame, and enzyme activities (RFU/min) were determined by calculating the slope between 10-70 seconds of the reaction. Results showing the activity of purified snow crab, blue swimming crab, DSN-5, DSN-6, DSN-1, and DSN-2 dsDNases in the presence of Mg²⁺ with or without calcium are shown in FIG. 8. All enzymes exhibit increased activity in the presence of Calcium (FIG. 8).

### Example 5: DNase Activity in the Presence of NaCl

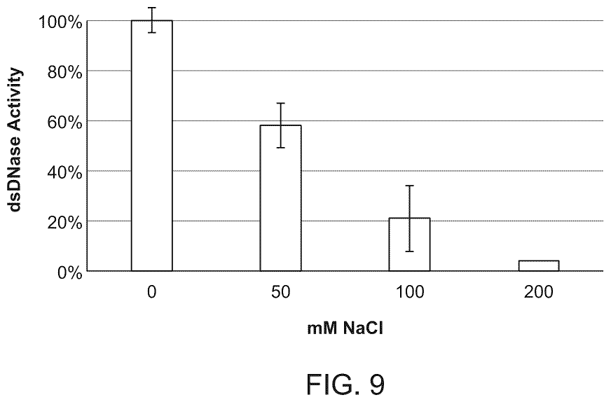

DNase activity was measured by monitoring the increase in fluorescence when a dsDNA hairpin substrate (5 μM) prepared according to Example 3 is cleaved by a purified dsDNase in 1× Reaction Buffer (10 mM Tris-HCl, pH 7.6, 2.5 mM MgCl₂, 0.5 mM CaCl₂), 100 μg/ml recombinant albumin) with 0, 50, 100, or 200 mM NaCl at 30 C in a black half-area 96-well plate (Costar 3694). Fluorescence was measured every 10 seconds for a total of 2 minutes. Activity was measured by monitoring the increase in fluorescence as the substrate is cleaved. Fluorescence increased linearly over this time frame, and enzyme activities (RFU/min) were determined by calculating the slope between 10-70 seconds of the reaction. Results are shown in FIG. 9. DSN-2 retained over half of its peak activity in the presence of 50 mM and about 20% of its activity in the presence of 100 mM NaCl.

### Example 6: DNase Activity Over a Range of Temperatures

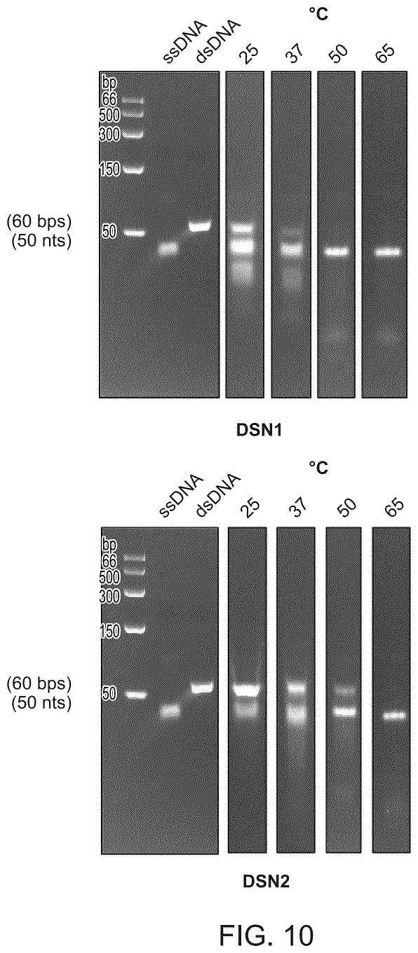

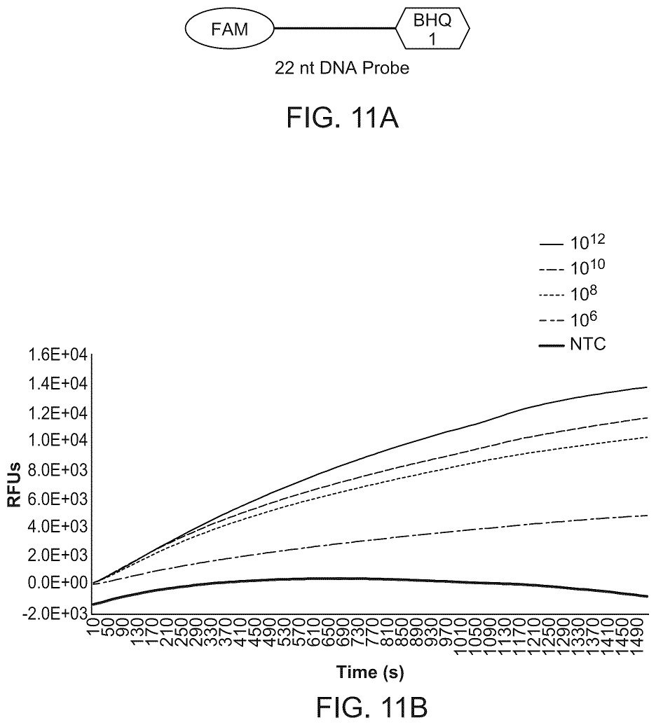

A mixture of 20 pmol of dsDNA (FIG. 2C) and 20 pmol of ssDNA (FIG. 2D) was incubated with either DSN1 or DSN2 for 1 minute (a, 25, 37, 50 or 65° ° C. in 1× Mg²⁺ Reaction Buffer (10 mM Tris-HCl, pH 7.9, 50 mM NaCl, 10 mM MgCl₂, 100 μg/ml recombinant albumin) and then immediately run on a 4% E-gel EX gel (Invitrogen) stained with Sybr Gold. RESULTS ARE shown in FIG. 10, in which lanes are as follows: (1) dsDNA PCR Marker, NEB #N3234, (2) ssDNA, (3) dsDNA (4) 25° C. (5) 37° C. (6) 50° ° C. (7) 65° C.

### Example 7: Non-Naturally Occurring dsDNases

Maltose binding protein fusion constructs with non-naturally occurring dsDNases were isolated and purified using Amylose resin. Purified Proteins are confirmed by SDS-PAGE analysis and enzymatic activities were evaluated with M13 ssDNA and supercoiled pUC19 (sc-pUC19) dsDNA or M13 ssDNA and 100 bp dsDNA ladder (NEB #N3231). Each reaction with the former pair of substrates contained 1.5 μg of M13 DNA, 0.5 μg of pUC19 DNA, 20 mM Tris-HCl (pH 8.0) and 10 mM MgCl₂ and was incubated at 65° C. for 60 minutes. Each reaction with the latter pair of substrates contained 1.5 ug of M13 ssDNA, 0.5 ug of 100 bp DNA ladder, 20 mM Tris-HCl (pH 8.0) and 10 mM MgCl₂ and was incubated at 65° C. for 60 minutes. Results are shown in Table 1.

TABLE 1
dsDNase Activity

| Enzyme | M13 ssDNA | pUC19 dsDNA | 100 bp dsDNA ladder |
|---|---|---|---|
| DSN-2 | − | +++++ | +++++ |
| DSN-12 | − | +++ | + |
| DSN-13 | − | ++++ | ++ |

### Example 8: Selective Cleavage of the DNA Strand of a DNA/RNA Heteroduplex

An in vitro transcription reaction (50 μl) was treated with 2 U DNase I-XT for 15 minutes at 37° C. to digest Cluc template DNA. The RNA was purified using the Monarch® RNA Cleanup Kit (500 μg, NEB #T2050) and eluted in nuclease-free water (50 μl). Varying amounts (from 10⁶-10¹² copies) of Cluc mRNA were mixed with a 5′ fluorescently labeled/3′ quenched 22 nt DNA probe (SEQ ID NO:30; 0.2 μM final concentration, Tm) 61.8° ° C. in NEBuffer r1.1. Duplex DNase (2U) was added, the reaction incubated at 58° C. and the relative fluorescence was monitored every 10 seconds in a BioRad CFX Touch™ qPCR instrument. The increase in fluorescence observed over time (which is not observed in the non-template control) demonstrates the ability of Duplex DNase to cleave the fluorescently labeled DNA strand of a DNA:RNA hybrid (probe:Cluc RNA). Moreover, the cleavage of the DNA probe when hybridized to the template RNA increases over time and is dose-dependent on the amount of Cluc template RNA present.

What is claimed is:

1. An engineered dsDNase having an amino acid sequence that is at least 95% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, or SEQ ID NO:13.

2. An engineered dsDNase according to claim 1, wherein the engineered dsDNase cleaves each strand of a DNA:DNA duplex and only the DNA strand of a DNA:RNA heteroduplex and does not cleave ssDNA.

3. An engineered dsDNase according to claim 2, wherein the engineered dsDNase comprises one or more bound magnesium cations.

4. An engineered dsDNase according to claim 1, wherein the amino acid sequence is at least 95% identical to SEQ ID NO: 1 or SEQ ID NO:2.

5. An engineered dsDNase according to claim 1, wherein the dsDNase in the presence of 50 mM NaCl has at least 50% of its peak activity in the absence of NaCl.

6. An engineered dsDNase according to claim 1, wherein the dsDNase in the presence of 100 mM NaCl has at least 20% of its peak activity in the absence of NaCl.

7. A composition comprising an engineered dsDNase according to claim 1 and one or more salts, wherein the composition has a total salt concentration of at least 50 mM.

8. A composition comprising an engineered dsDNase according to claim 1 and one or more DNA hydrolysis products.

9. A composition according to claim 8 further comprising one or more enzymes other than the dsDNase, one or more buffering agents, one or more detergents, one or more glass-forming agents, one or more salts, one or more stabilizers, one or more DNA:DNA duplexes, one or more DNA:RNA duplexes, or combinations thereof.

10. A fusion protein comprising a single polypeptide chain, the single peptide chain comprising:
(a) a dsDNase according to claim 1; and
(b) at least one of an affinity tag, a secretion signal, and a linker.

11. An engineered dsDNase comprising an amino acid sequence at least 97% identical to the amino acid sequence of SEQ ID NO: 19, SEQ ID NO: 28, or SEQ ID NO: 29.

12. An engineered dsDNase according to claim 11, wherein the amino acid sequence is identical to SEQ ID NO:19, SEQ ID NO: 28, or SEQ ID NO: 29.

13. A method for depleting a target sequence from a population of duplex DNA molecules, the population comprising at least one copy of a DNA strand having the target sequence, the method comprising:
(a) hybridizing to the at least one strand a polynucleotide having a sequence complementary to the target sequence to form a target duplex, and
(b) contacting the target duplex with a dsDNase according to claim 1 to form a target sequence depleted population of duplex DNA molecules and DNA hydrolysis products.

14. A method according to claim 13, wherein the polynucleotide having a sequence complementary to the target sequence is an oligonucleotide probe.

15. A method according to claim 13, wherein the polynucleotide having a sequence complementary to the target sequence comprises RNA.

16. A method according to claim 13, wherein the polynucleotide having a sequence complementary to the target sequence comprises DNA.

17. A method according to claim 16, wherein the polynucleotide having a sequence complementary to the target sequence is a hairpin probe comprising, in a 5′ to 3′ direction, a fluorophore, a first hairpin stem sequence, the sequence complementary to the target sequence, a second hairpin stem sequence complementary to the first hairpin stem sequence, and a quencher corresponding to the fluorophore.

18. A method according to claim 17, wherein the DNA hydrolysis products comprise the fluorophore and the quencher, wherein the fluorophore is sufficiently separated from the quencher to be fluorescently active.

19. A method according to claim 13, wherein the target sequence is a repetitive sequence present in the population of duplex DNA molecules.

20. A method according to claim 19, wherein (a) further comprises denaturing and reannealing the population of duplex DNA molecules and each target duplex comprises hybridized repetitive sequences.

21. A method for hydrolyzing DNA comprising contacting (a) a composition comprising DNA and optionally RNA, and (b) a dsDNase according to claim 1 to form a reaction mixture comprising DNA hydrolysis products.

22. A method according to claim 21, wherein the reaction mixture has a total salt concentration of 0 mM to 150 mM.

23. A method according to claim 21, wherein the composition comprises RNA and the reaction mixture comprises at least 90% of the RNA that was in the composition.

24. A method according to claim 21, wherein the reaction mixture comprises less than 10% of the DNA that was in the composition.

25. A method according to claim 21, wherein the reaction mixture further comprises a DNase I buffer or a high magnesium buffer.

26. A method according to claim 21, wherein the composition comprises one or more DNA:DNA duplexes, one or more RNA:DNA duplexes, or both one or more DNA:DNA duplexes and one or more RNA:DNA duplexes.

27. A method according to claim 26, wherein the duplex DNA comprises no more than one mismatch per 10 paired nucleotides.

28. A method according to claim 26, wherein the duplex DNA comprises repetitive DNA sequences.

29. A composition comprising a dsDNase having an amino acid sequence that is at least 85% identical to SEQ ID NO:14, SEQ ID NO:15, SEQ ID NO:16, SEQ ID NO: 17, or SEQ ID NO: 18, wherein the composition is free of one or more of a DNA polymerase, an RNA polymerase, and a protease.

30. A method for depleting a target sequence from a population of duplex DNA molecules, the population comprising at least one copy of a DNA strand having the target sequence, the method comprising:
(a) hybridizing to the at least one strand a polynucleotide having a sequence complementary to the target sequence to form a target duplex, and
(b) contacting the target duplex with a dsDNase having an amino acid sequence that is at least 85% identical to SEQ ID NO:14, SEQ ID NO:15, SEQ ID NO:16, SEQ ID NO:17, or SEQ ID NO: 18 to form a target sequence depleted population of duplex DNA molecules and DNA hydrolysis products.

31. A method according to claim 30, wherein the polynucleotide having a sequence complementary to the target sequence is an oligonucleotide probe.

32. A method according to claim 30, wherein the polynucleotide having a sequence complementary to the target sequence comprises RNA.

33. A method according to claim 30, wherein the polynucleotide having a sequence complementary to the target sequence comprises DNA.

34. A method according to claim 33, wherein the polynucleotide having a sequence complementary to the target sequence is a hairpin probe comprising, in a 5′ to 3′ direction, a fluorophore, a first hairpin stem sequence, the sequence complementary to the target sequence, a second hairpin stem sequence complementary to the first hairpin stem sequence, and a quencher corresponding to the fluorophore.

35. A method according to claim 34, wherein the DNA hydrolysis products comprise the fluorophore and the quencher, wherein the fluorophore is sufficiently separated from the quencher to be fluorescently active.

36. A method according to claim 30, wherein the target sequence is a repetitive sequence present in the population of duplex DNA molecules.

37. A method according to claim 36, wherein (a) further comprises denaturing and reannealing the population of duplex DNA molecules and each target duplex comprises hybridized repetitive sequences.

38. A method for hydrolyzing DNA comprising contacting (a) a composition comprising DNA and optionally RNA, and (b) a dsDNase having an amino acid sequence that is at least 85% identical to SEQ ID NO:14, SEQ ID NO:15, SEQ ID NO:16, SEQ ID NO:17, or SEQ ID NO:18 to form a reaction mixture comprising DNA hydrolysis products.

39. A method according to claim 38, wherein the reaction mixture has a total salt concentration of 0 mM to 150 mM.

40. A method according to claim 38, wherein the composition comprises RNA and the reaction mixture comprises at least 90% of the RNA that was in the composition.

41. A method according to claim 38, wherein the reaction mixture comprises less than 10% of the DNA that was in the composition.

42. A method according to claim 38, wherein the reaction mixture further comprises a DNase I buffer or a high magnesium buffer.

43. A method according to claim 38, wherein the composition comprises one or more DNA:DNA duplexes, one or more RNA:DNA duplexes, or both one or more DNA:DNA duplexes and one or more RNA:DNA duplexes.

44. A method according to claim 43, wherein the duplex DNA comprises no more than one mismatch per 10 paired nucleotides.

45. A method according to claim 43, wherein the duplex DNA comprises repetitive DNA sequences.

46. A composition comprising:
a means for selectively cleaving a DNA strand of a DNA:RNA duplex; and
a non-naturally occurring buffer,
wherein the selective cleavage comprises cleaving ≥90% of the DNA strands of a DNA:RNA duplex at least once and cleaving ≤10% of the RNA strands even once.

47. A composition according to claim 46, wherein the means for selectively cleaving a DNA strand of a DNA:RNA duplex comprises an engineered dsDNase having an amino acid sequence that is (a) at least 95% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO: 12, or SEQ ID NO: 13, and (b) optionally 100% identical to SEQ ID NO:32.

48. A composition according to claim 46, wherein the means for selectively cleaving a DNA strand of a DNA:RNA duplex comprises an engineered dsDNase having an amino acid sequence that is (a) at least 95% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO: 12, or SEQ ID NO:13, and (b) optionally 100% identical to SEQ ID NO:31.

49. A composition according to claim 46, further comprising one or more enzymes other than the dsDNase, one or more detergents, one or more glass-forming agents, one or more salts, one or more stabilizers, one or more DNA:DNA duplexes, one or more DNA:RNA duplexes, or combinations thereof.

50. A composition according to claim 46, wherein the non-naturally occurring buffer is a storage buffer or a reaction buffer.


SEQUENCE LISTING (32 sequences)

SEQ ID NO: 1 - protein, 379 aa, synthetic construct
  REGION 1..379: note = Synthetic construct
  REGION 1..379: note = MISC_FEATURE - DSN-1 (Example dsDNase)
QDCTWDKDNDFPEYPPLLLDEQMELIRPVMEGDLRIVRVAAGAKLTVACPGSLVTNVGSETAEGTCGGGDILVIGNTEWRLEELGCTKKVMETIQRNLASCGGSGEGQLESIGFQIGTDFYELIRVCFEPKAQTTLYAEHVIHGANIAAKDIDSSRPSFKTSSGFFSVSMSTVYAQKSQLELMTKLLGEEGLANTIIDTHKQLYFAKGHMAPDADFVTEAEQDATYYYINAVPQWQAFNNGNWKYLEFATRDLASAHGSDMRIYSGGWGVLELNDINNNPVRILLGLSENKEVVPAPAVTWKVIYEESSGKAAALVGVNNPHLTNAPTPLCSDLCSNLSWIDFDVEDMAHGYTYCCTVDDLRAAIPHVPDLGPVELLDK

SEQ ID NO: 2 - protein, 381 aa, synthetic construct
  REGION 1..381: note = Synthetic construct
  REGION 1..381: note = MISC_FEATURE - DSN-2 (Example dsDNase)
QECVWNKDADFPEYPPIILDASFEIVRPVAEGELRIVRVSAGAKLTLACPGSEITNLGTVAVDVICGGGDLLVVDGTEWRMDELGCSKKDKESIHRNLGSCGDGGVGVFEGIGFEIFGPESFYELIRVCFEPKAETTLYSEHVVRGANIAAKDIDSSRPSFKSSTGFFTVSMSTIYTQNSQLALMKTLLGDDDLANTIINPHEQLYFARGHLAPDADFVTEAEQDATYYYINAVPQWQAFNNGNWKYLEFATRDLAEAHGTDLTIYTGGWGVLALDDINGNPVEIYLGLTENEMVVPAPAITWKVVYEESSSRAVGVVGVNNPHITSPPTPLCSDLCSSLSWIDFDVNDLGHGYTYCCTVDDLRAAIPHVPDLGSVGLLDK

SEQ ID NO: 3 - protein, 381 aa, synthetic construct
  REGION 1..381: note = Synthetic construct
  REGION 1..381: note = MISC_FEATURE - DSN-3 (Example dsDNase)
QECVWNKDTDFPEYPPIILDASFEIVRPVAEGEVRVVRVSAGAKLTLACPGSEITNLGTTAVDVTCGGGDLLVVDGTEWRMDELGCSKKDKESIHRNLGSCGDGGVGVFEGIGFEIFGSDSFYELIRVCFEPKAETTLYSEHVLHGANIAAKDIDSSRPSFKSSTGFFTVSMSTCYTQNSQLALMKTLLGDDDLANTIINPHEQLYFAKGHLAPDADFVTEAEQDATYYYINAVPQWQAFNNGNWKYLEFATRDLAEAHGTDLTIYTGGWGVLTLDDINGNPVEIYLGLTEDEMVVPAPAITWKVVYEESSSRAVGVVGVNNPHITSPPTPLCSDLCSSLSWIDFDVNDLGHGYTYCCTVDDLRAAIPHVPDLGSVGLLDK

SEQ ID NO: 4 - protein, 381 aa, synthetic construct
  REGION 1..381: note = Synthetic construct
  REGION 1..381: note = MISC_FEATURE - DSN-4 (Example dsDNase)
QDCVWDKDTDFPEYPPLLLDSSFEMVRPVLEGSVRMVRVSAGSKVTLACPGSEITNLGVTAVEATCAGGTLLAIDGTEWTLAELGCSKKVKESIHRNLGSCGDGDVGVLESIGFEIFGSDSFYELIRVCFEPKAETTLYTQHVLHGANIAAKDIDSSRPSFKTSTGFFTVSMSTVYAQNSQLELMTTLLGDADLANTIIDPHKQFYFAKGHMSPDADFVTEAEQDATYYYINAVPQWQAFNNGNWKYLEFATRDLAEKHGTDLTIYSGGWGVLELDDINGNPVEIYLGLSEDETVVPAPAITWKVVHEESSNRAVAVVGVNNPHITSAPTPLCSDLCSSLSWIDFDVSDLAHGYTYCCTVDDLRAAIPHVPDLGNVGLLDD

SEQ ID NO: 5 - protein, 381 aa, synthetic construct
  REGION 1..381: note = Synthetic construct
  REGION 1..381: note = MISC_FEATURE - DSN-5 (Example dsDNase)
SDCVWNKDLDFPQYPPLIIDSSLEFVLPVLEGGNRVVRVAAGDTVTLACPGNELTNLGQVEAEARCLSSGLLAIDGSEWDLASLGCSDDVKETIFRNLGTCGAGDIGILHGIGFEIFSLNNYKEIIRVCFEPSSETTLYTEHVIHGANIAAKDIDSSRPSFKTSTGFFSVSMSTVYSQNSQLQLMIDLLGDEDLANTIIDPSKQLYFAKGHMSPDADFVTVANQDATYYYINALPQWQAFNNGNWKYLEYATRDLAEKKGRDLRVYSGGWGVLELDDINGNPVEVFLGLTEGKKVVPAPAVTWKVVHDESTNCAAAVVGVNNPHLTSAPSTLCEDLCSSLSWIDFDVSDLAHGYTYCCSVADLRAAVPHVPDLGDVCLLTD

SEQ ID NO: 6 - protein, 381 aa, synthetic construct
  REGION 1..381: note = Synthetic construct
  REGION 1..381: note = MISC_FEATURE - DSN-6 (Example dsDNase)
QDCAWDKDTDFPENPPLLLDSSLQMILPVLEGSLRMVRVPSGSEITLACPGSTISALGLEAVEGTCAGGTLISIDGTDYSLSELGCSNKVSETIQKNLGSCGDGGIGTLHAIGFDIATTGSFYELIRVCFDHAAETTLYTQHILHGANIAAKDVDSSRPSFKTSTGFFTVSMNTVYTQASQLELMENLLGDADLANQIIDPSQQLYFAKGHMSPDADFVTIAEQDATYYFINAVPQWQAFNNGNWKYLEYATRDLAESHGTDLTVYSGGWGVLELDDINGNPVEIYLGLSEDKTVVPAPALTWKVVHEESTNRAAAVVGINNPHITSAPTPLCTDICSSLSWIDFDFGDLTRGYTYCCTVDDLRAAIPHVPDLGNVGLLDS

SEQ ID NO: 7 - protein, 385 aa, synthetic construct
  REGION 1..385: note = Synthetic construct
  REGION 1..385: note = MISC_FEATURE - DSN-7 (Example dsDNase)
EDCVWNKDTDYPDNPPLILDGYFKLVLPVMKDTTREVRVPSGSIVTLACPGTGNSLAQLGEVAVQATCVGGVMLDVNGTEMPLSTLGCSKKIRSTIKKNLGSCGAGDLGQLHIIGFQIADKFYEQILVCFDHNAQTTLYTRHVIHGANIAAKDIDSSRPSFKTSRGFFNVSMSTVYTQNSQLQLLINLLGDEDLANTIFDTSKYYFAKGHLSPDADFVTEAEQDATYYYINAVPQWQAFNNGNWKFLEYATRDLAEQKGRDLRVYSGGWGVLELDDINGNPVEVFLGLTEGKAVVPAPAVTWKVVHDEYSNCAVAVVGVNNPHLTSAPTTLCEDLCSSLSWIDFDVSDLTRGYTYCCTVADLRAAVPHVPDLGDVCLLTNNGPGC

SEQ ID NO: 8 - protein, 381 aa, synthetic construct
  REGION 1..381: note = Synthetic construct
  REGION 1..381: note = MISC_FEATURE - DSN-8 (Example dsDNase)
QDCVWNKDADFPEYPPLILDSSFEFVLPVLEGDLRVVRVSAGATLTLACPGSEITNLGTVAVDAICLGGDLLVVDGTEWRLAELGCSKKDKESIHRNLGSCGDGGVGVLEGIGFEIFGPESFYELIRVCFEPKAETTLYSEHVVHGANIAAKDIDSSRPSFKTSTGFFSVSMSTVYSQNSQLALMKTLLGDEDLANTIINPHKQLYFAKGHLAPDADFVTEAEQDATYYYINAVPQWQAFNNGNWKYLEFATRDLAEKHGTDLTVYTGGWGVLELDDINGNPVEIYLGLTENKMVVPAPAVTWKVVYEESTSRAAAVVGVNNPHITSAPTPLCSDLCSSLSWIDFDVNDLGHGYTYCCTVDDLRAAIPHVPDLGNVGLLDK

SEQ ID NO: 9 - protein, 379 aa, synthetic construct
  REGION 1..379: note = Synthetic construct
  REGION 1..379: note = MISC_FEATURE - DSN-9 (Example dsDNase)
QDCAWDKDNDFPEYPPLLLDESMELIRPVEEGSLRMVRVAAGAKITVACPGSLVANVGLEAVEGTCGGGDILVIGSTEWRLSELGCTKKVSETIQRNLASCGGSGEGQLESIGFQIGTDFYELIRVCFEPKAQTTLYAQHVLHGANIAAKDIDSSRPSFKTSSGFFTVSMSTVYAQKSQLELMIKLLGDEGLANTIIDTHKQLYFAKGHMAPDADFVTEAEQDATYYYINAVPQWQAFNNGNWKYLEFATRDLATAHGSDMTIYSGGWGVLELDDINNNPVRILLGLSEDKEVVPAPAVTWKVVHEESSGRAAAVVGVNNPHLTSAPTPLCADLCSNLSWIDFDVGDLAHGYTYCCTVDDLRAAIPHVPDLGPVGLLDK

SEQ ID NO: 10 - protein, 379 aa, synthetic construct
  REGION 1..379: note = Synthetic construct
  REGION 1..379: note = MISC_FEATURE - DSN-10 (Example dsDNase)
QDCAWDKDNDFPEYPPLILDENMELIRPVEEGSVRMVRVPAGAKLTVACPGNEVANVGEVAIEATCGGGDLLVQGSTEWRLVELGCTKKVKESIHRNLASCGGGGTGQLESIGFQIGGDFYDLIQVCFEPKAQTTLYAQHVVHGANIAAKDIDSSRPSFKTSTGFFSVSMSTVYAQKSQLELMITLLGDEDLANSIIDTHKQFYFAKGHMAPDADFVTEAEQDATYYYINAVPQWQAFNNGNWKYLEFATRDLAEAHGTDMTIYSGGWGVLELDDINNNPVRILLGLYEGEEVVPAPAVTWKVVHEEGSGRAAAVVGVNNPHLTSAPTPLCADLCDNLTWIDFDVNDLAHGYTYCCTVDDLRAAVPHVPDLGPVDLLDK

SEQ ID NO: 11 - protein, 381 aa, synthetic construct
  REGION 1..381: note = Synthetic construct
  REGION 1..381: note = MISC_FEATURE - DSN-11 (Example dsDNase)
QDCAWDKDNDFPEYPPLILDENFELVRPVLEGSVRMVRVPAGAKLTLACPGSEITNLGTTAVEATCGGGDLLVIDGTEWRMAELGCTKKVKETIHRNLGSCGGGGVGELHSIGFEIYSSGSFYELIRVCFEPKAETTLYTEHVIHGANIAAKDIDSSRPSFKTSTGFFSVSMSTVYAQKSQLELMTTLLGDEDLANSIIDPHKQFYFAKGHMAPDADFVTEAEQDATYYYINAVPQWQAFNNGNWKYLEFATRDLAEAHGTDMTIYSGGWGVLELDDINNNPVRILLGLTEGEEVVPAPAVTWKVVHEEGSGRAAAVVGVNNPHITSAPTPLCSDLCDNLTWIDFDVNDLAHGYTYCCTVDDLRAAIPHVPDLGPVDLLDK

SEQ ID NO: 12 - protein, 381 aa, synthetic construct
  REGION 1..381: note = Synthetic construct
  REGION 1..381: note = MISC_FEATURE - DSN-12 (Example dsDNase)
QECMWNKDADFPEYPPIILDASFEIVRPVMEGEARVVRVSAGAKLTLACPGSEITNLGTVAVDLQCGGGDLLVVDGTEWRMDELGCSKKDKESIHRNLGSCGDGGVGVFEGIGFEIFGPESFYELIRVCFEPKAETTLYSEHVLRGANIAAKDIDSSRPSFKSSTGFFTVSMSTIYTQNSQLASMKILLGDDDLANAILNPQEQYYFAKGHLAPDADFVTEAEQDATYYYINAVPQWQAFNNGNWKYLEFATRDLAEAHGTDLTIYTGGWGVLTLDDINGNPVEIYLGLTENEMVVPAPAITWKVIYEESSSRAVGVVGVNNPHITSPPTPLCSDLCSSLAWIDFDVNDLGHGYTYCCTVDDLRAAIPHVPDLGNVGLLDK

SEQ ID NO: 13 - protein, 381 aa, synthetic construct
  REGION 1..381: note = Synthetic construct
  REGION 1..381: note = MISC_FEATURE - DSN-13 (Example dsDNase)
QECMWNKDTDFPEYPPIILDASLEIVRPVMEGELRVVRVSAGAQLTLACPGSEITNLGTTAADVICGGGNLLVVDGTEWRLDELGCSKKDKESIHRNLGSCGDGGVGVFEGIGFEIFGSDSFYELIRVCFEPKAETTLYSEHVLHGANIAAKDIDSSRPSFKSSTGFFTVSMSTIYTQNSQLALMKILLGDDDLANTILNPQEQLYFARGHLAPDADFVTEAEQDATYYYINAVPQWQAFNNGNWKYLEFATRDLAESHSTDLTIYTGGWDVLTLDDINGNPVEIYLGLTEDEMVVPAPAVTWKVVYEESSSRAVGVVGVNNPHITSPPTPLCSDLCSSLAWIDFDVDDLGHGYTYCCTVDDLRAAIPHVPDLGSVGLLDK

SEQ ID NO: 14 - protein, 381 aa, Chionoecetes opilio
  REGION 1..381: note = MISC_FEATURE - Snow crab (Example dsDNase)
SDCVWNRDSDFPVYSPLILDSSFDFVLPVEEDGNKIVRIAAGATVTLACPGNEIASLHQVEAEARCLDNGLLAIDNSEWDLASLGCARPVKETIFRDLGTCGAEDIGTLHAIGFEIVSLGKYKEIIRVCFEPSSETTLFTEHVIHGANIAAKDIDTSRPSFRTSSGFFSISMIKAYSQSSQLVLMTNLLGDEDLALSVIDIHKQLYFAKGHMSPDADFVLMANQDASYYYINALPQWQVFNNGNWRNLEYATRDLAEKKGRDLRVISGGWGILELNDINGNPVEIFLGLIDDKKVVPAPAITWKVVYDESTNCAAAVVGVNNPFLTTAPRKLCEDLCSSLSWIDFDVGDLAHGYTYCCSVKDLRASVPHVPDLGDVCLLTD

SEQ ID NO: 15 - protein, 379 aa, Acetes chinensis
  REGION 1..379: note = MISC_FEATURE - Northern mauxia shrimp (Example dsDNase)
QDCAWDKDNDFPEYPPLLLDENMELIRPVEEGSVRMVRVAAGAKVTVACPGNQVANVGEEVIQTTCGGGDILVQGSTEWRLLELGCTKKVSESIQRNLASCGGSGEGQLESIGFQIGTDFYDLIQVCFEPKAQTTLYAQHVLHGANIAAKDIDSSRPSFKSSSGFFTVSMSTVYAQKSQLELMIKLLGDEGLANTIIDTHKQLYFAKGHMAPDADFVTEAEQDATYYYINAVPQWQAFNNGNWKYLEFATRDLATAHGSDMTIYSGGWGVLELDDINNNPARILLGLYEDEEVVPAPAVTWKVVHEEGSGRAVAVVGVNNPHLTSPPTPLCADLCGNLTWIDFDVGDLAHGYTYCCTVDDLRAAVPHVPDLGPVDLLDK

SEQ ID NO: 16 - protein, 379 aa, Acetes chinensis
  REGION 1..379: note = MISC_FEATURE - Northern mauxia shrimp (Example dsDNase)
QDCAWDKDNDFPEYPPLLLDENMELIRPVEEGSVRMVRVDAGAKVTVACPGNQVANVGEELIQTTCGGGDILVQGSTEWQLLELGCTKKVSESIQRNLASCGGSGEGQLESIGFQIGTDFYDLIQVCFEPKAQTTLYARHVLHGANIAAKDIDSSRPSFKTSSGFFTVSMSTVYAQKSQLELMIKLLGDEGLANTIIDTHKQLYFAKGHMAPDADFVTEAEQDATYYYINAVPQWQAFNNGNWKYLEFATRKLAATHGSDMTIYSGGWDVLELNDVNNNPARILLGLYKNQEVVPAPAITWKVVHEEGSGRAVAVVGVNNPHLINPPTPLCTNLCGNLTWIDFDVSDLAHGYTYCCTVDDLRAAVPHVPDLGPVDLLDK

SEQ ID NO: 17 - protein, 381 aa, Penaeus monodon
  REGION 1..381: note = MISC_FEATURE - Giant tiger prawn (Example dsDNase)
QECVWNKDADFPEYPPIILDASFEIVRPVMEGELRIVRVSAGAQLTLACPGSEITNLGTVAADLICGGGDLLVVDGTEWRLDELGCTVKDKESIHRNLGSCGDGGVGVFEGIGFEIFGPESFYELIRVCFEPKAETTLYSEHVVRGANIAAKDIDSSRPSFKSSTGFFTVSMSTIYTQNSQLALMKTLLGDDDLANTILNPQEQLYFARGHLAPDADFVTEAEQDATYYYINAVPQWQAFNNGNWKYLEFATRDLAEAHGTDLTIYTGGFDVLALDDINANPVEIYLGLTENEMVVPAPAVTWKVIYEESSSRAVGVVGVNNPHITSPPTPLCSDLCSSLSWIDFNVDDLGHGYTYCCTVDDLRAAIPHVPDLGNVGLLDK

SEQ ID NO: 18 - protein, 381 aa, Portunus pelagicus
  REGION 1..381: note = MISC_FEATURE - Blue swimming crab (Example dsDNase)
SDCMWDKDKDFPRYPPLILDSNYEFVLPVEENGNRVVRLALGEIVTLACPGDELENLHLVEAKARCLEDGLLMVEDSVWDMASLGCGDDVKEMILRDQGACGAGDIGVLHEIGFEVLSAERFKKVLRVCFDPASETTLYTEHLIHGASIAAKDIDSSRPSFKTSTGFFSVSMSSVYSQSSQLELMIKLLGDEDLAHQIIDTHKQLYFAKGHMSPDADFVIMDNQDATYYYINAVPQWQAFNNGNWKYLEFATRDLAEKKKRDLRVYSGGWGVLELDDINGNPVEVFLGLAQEKKVVPAPAVTWKVVHDEVTNCAAAIVGVNNPHLTSAPSTLCEDLCSSLPWIDFDVADLEHGYTYCCSVADLRAAVPHVPDLGEVCLLTD

SEQ ID NO: 19 - protein, 396 aa, synthetic construct
  REGION 1..396: note = Synthetic construct
  REGION 1..396: note = MISC_FEATURE - Snow crab variant (Example dsDNase)
  REGION 5..385: note = MISC_FEATURE - Example dsDNase (Chionoecetes opilio)
EAEASDCVWNRDSDFPVYSPLILDSSFDFVLPVEEDGNKIVRIAAGATVTLACPGNEIASLHQVEAEARCLDNGLLAIDNSEWDLASLGCARPVKETIFRDLGTCGAEDIGTLHAIGFEIVSLGKYKEIIRVCFEPSSETTLFTEHVIHGANIAAKDIDTSRPSFRTSSGFFSISMIKAYSQSSQLVLMTNLLGDEDLALSVIDIHKQLYFAKGHMSPDADFVLMANQDASYYYINALPQWQVFNNGNWRNLEYATRDLAEKKGRDLRVISGGWGILELNDINGNPVEIFLGLIDDKKVVPAPAITWKVVYDESTNCAAAVVGVNNPFLTTAPRKLCEDLCSSLSWIDFDVGDLAHGYTYCCSVKDLRASVPHVPDLGDVCLLTDNSAVDHHHHHH

SEQ ID NO: 20 - protein, 4 aa, synthetic construct
  REGION 1..4: note = Synthetic construct
  REGION 1..4: note = MISC_FEATURE - N-terminal leader
  SITE 4: note = A or AAE
EAEA

SEQ ID NO: 21 - protein, 11 aa, synthetic construct
  REGION 1..11: note = Synthetic construct
  REGION 1..11: note = MISC_FEATURE - C-terminal linker and His6 tag
NSAVDHHHHHH

SEQ ID NO: 22 - DNA, 24 bp, synthetic construct
  misc_feature 1..24: note = Synthetic construct
  misc_feature 1..24: note = Primer
  source 1..24: mol_type = other DNA
gctcattccaattccttctattag

SEQ ID NO: 23 - DNA, 23 bp, synthetic construct
  misc_feature 1..23: note = Synthetic construct
  misc_feature 1..23: note = Primer
  source 1..23: mol_type = other DNA
gagctccaatcaagcccaataac

SEQ ID NO: 24 - DNA, 35 bp, synthetic construct
  misc_feature 1..35: note = Synthetic construct
  misc_feature 1..35: note = hairpin dsDNase substrate
  misc_feature 1: note = 5' FAM label
  misc_feature 35: note = 3' Black Hole Quencher-1
  source 1..35: mol_type = other DNA
tctaagccgtgtacattttttgtacacggcttaga

SEQ ID NO: 25 - DNA, 15 bp, synthetic construct
  misc_feature 1..15: note = Synthetic construct
  misc_feature 1..15: note = single stranded DNase substrate
  misc_feature 1: note = 5' FAM label
  misc_feature 15: note = 3' Black Hole Quencher-1
  source 1..15: mol_type = other DNA
tgaagtaatctgtta

SEQ ID NO: 26 - DNA, 60 bp, synthetic construct
  misc_feature 1..60: note = Synthetic construct
  misc_feature 1..60: note = linear dsDNase substrate
  misc_feature 1: note = 5' FAM label
  source 1..60: mol_type = other DNA
tggagattttgatcacggtaaccgatcagaatgacaacaagcccgaattcacccaggagg

SEQ ID NO: 27 - DNA, 50 bp, synthetic construct
  misc_feature 1..50: note = Synthetic construct
  misc_feature 1..50: note = single stranded DNase substrate
  misc_feature 1: note = 5' ROX label
  source 1..50: mol_type = other DNA
caccttcaacccagttcctagtcgtagcctttatccgagattcagcaata

SEQ ID NO: 28 - protein, 394 aa, synthetic construct
  REGION 1..394: note = Synthetic construct
  REGION 1..394: note = MISC_FEATURE - DSN-1 (Example dsDNase)
  REGION 5..383: note = MISC_FEATURE - Example dsDNase
EAEAQDCTWDKDNDFPEYPPLLLDEQMELIRPVMEGDLRIVRVAAGAKLTVACPGSLVTNVGSETAEGTCGGGDILVIGNTEWRLEELGCTKKVMETIQRNLASCGGSGEGQLESIGFQIGTDFYELIRVCFEPKAQTTLYAEHVIHGANIAAKDIDSSRPSFKTSSGFFSVSMSTVYAQKSQLELMTKLLGEEGLANTIIDTHKQLYFAKGHMAPDADFVTEAEQDATYYYINAVPQWQAFNNGNWKYLEFATRDLASAHGSDMRIYSGGWGVLELNDINNNPVRILLGLSENKEVVPAPAVTWKVIYEESSGKAAALVGVNNPHLTNAPTPLCSDLCSNLSWIDFDVEDMAHGYTYCCTVDDLRAAIPHVPDLGPVELLDKNSAVDHHHHHH

SEQ ID NO: 29 - protein, 393 aa, synthetic construct
  REGION 1..393: note = Synthetic construct
  REGION 1..393: note = MISC_FEATURE - DSN-2 (Example dsDNase)
  REGION 7..387: note = MISC_FEATURE - Example dsDNase
EAEAAEQECVWNKDADFPEYPPIILDASFEIVRPVAEGELRIVRVSAGAKLTLACPGSEITNLGTVAVDVICGGGDLLVVDGTEWRMDELGCSKKDKESIHRNLGSCGDGGVGVFEGIGFEIFGPESFYELIRVCFEPKAETTLYSEHVVRGANIAAKDIDSSRPSFKSSTGFFTVSMSTIYTQNSQLALMKTLLGDDDLANTIINPHEQLYFARGHLAPDADFVTEAEQDATYYYINAVPQWQAFNNGNWKYLEFATRDLAEAHGTDLTIYTGGWGVLALDDINGNPVEIYLGLTENEMVVPAPAITWKVVYEESSSRAVGVVGVNNPHITSPPTPLCSDLCSSLSWIDFDVNDLGHGYTYCCTVDDLRAAIPHVPDLGSVGLLDKHHHHHH

SEQ ID NO: 30 - RNA, 1763 nt, synthetic construct
  source 1..1763: mol_type = other RNA
gggagacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggcggccgcaataaaatatctttattttcattacatctgtgtgttggttttttgtgtgtctag

SEQ ID NO: 31 - protein, 383 aa, synthetic construct
XXCXWBXDXDXPXXXPJJJDXXXXXXXPVXXXXXXXVRXXXGXXXTXACPGXXXXXXXXXXXXXXXXCXXXXXJXXXXXXXXXXXLGCXXXXXXXIXXBXXXCGXXXXGXXXXIGFXXXXXXXXXXXJXVCFXXXXZTTLXXXHXXXGAXIAAKDXDXSRPSFXXSXGFFXXSMXXXYXQXSQLXXXXXLLGXXXLAXXXXBXXXXXYFAXGHXXPDADFVXXXXQDAXYYXINAXPQWQXFNNGNWXXLEXATRXLAXXXXXDXXXXXGGXXXLXLBDXNXNPXXXXLGLXXXXXVVPAPAXTWKVXXXEXXXXAXXXVGXNNPXJXXXPXXLCXBJCXXLXWIDFBXXDXXXGYTYCCXVXDLRAXXPHVPDLGXVXLLXX

SEQ ID NO: 32 - protein, 383 aa, synthetic construct
XXCXWXXDXDXPXXXPXXXDXXXXXXXPVXXXXXXXVRXXXGXXXTXACPGXXXXXXXXXXXXXXXXCXXXXXXXXXXXXXXXXXLGCXXXXXXXIXXXXXXCGXXXXGXXXXIGFXXXXXXXXXXXXXVCFXXXXXTTLXXXHXXXGAXIAAKDXDXSRPSFXXSXGFFXXSMXXXYXQXSQLXXXXXLLGXXXLAXXXXXXXXXXYFAXGHXXPDADFVXXXXQDAXYYXINAXPQWQXFNNGNWXXLEXATRXLAXXXXXDXXXXXGGXXXLXLXDXNXNPXXXXLGLXXXXXVVPAPAXTWKVXXXEXXXXAXXXVGXNNPXXXXXPXXLCXXXCXXLXWIDFXXXDXXXGYTYCCXVXDLRAXXPHVPDLGXVXLLXX